(19) United States
(12) Patent Application Publication
(10) Pub. No.: US 2025/0011763 A1
(43) Pub. Date: Jan. 9, 2025

(54) ACCURATE SEQUENCING LIBRARY GENERATION VIA ULTRA-HIGH PARTITIONING

(71) Applicant: Enumerix, Inc., Palo Alto, CA (US)

(72) Inventors: Eleen Yee Lam SHUM, San Carlos, CA (US); Hei Mun Christina FAN, Palo Alto, CA (US); Stephen P.A. FODOR, Palo Alto, CA (US)

(21) Appl. No.: 18/759,345

(22) Filed: Jun. 28, 2024

Related U.S. Application Data
(63) Continuation of application No. PCT/US2023/010039, filed on Jan. 3, 2023.
(60) Provisional application No. 63/296,261, filed on Jan. 4, 2022.

(51) Int. Cl. C12N 15/10; C12Q 1/6806; C12Q 1/686

(52) U.S. Cl. CPC C12N 15/1093; C12Q 1/6806; C12Q 1/686

(57) ABSTRACT

The disclosure provides compositions, methods, and systems for extremely accurate generation of nucleic acid libraries, without use of bulk amplification methods. Accurate library preparation is achieved in a rapid manner, with respect to sample partitioning and amplification in a manner that achieves high performance in relation to low levels of amplification bias and low levels of artifact/chimeric sequence generation. Implementation of methods described also achieve library preparation with significantly reduced false positive rates, across a wide variety of applications.

### CROSS-REFERENCE

This application is a continuation of International Patent Application No. PCT/US2023/010039, filed Jan. 3, 2023, which claims the benefit of U.S. Provisional Application No. 63/296,261, filed Jan. 4, 2022, each of which applications is incorporated in its entirety herein by this reference.

### TECHNICAL FIELD

The disclosure generally relates to systems, methods, and compositions for library preparation.

### BACKGROUND

Next-generation sequencing (NGS) has become the dominant technology platform for high-complexity diagnostic tests. For these tests, the starting material often contains only trace amounts of representative DNA targets and requires amplification to generate sequencing libraries. Even though polymerase chain reaction (PCR) and other amplification processes can be used to amplify trace amounts of DNA for subsequent analyses, amplification processes harbor intrinsic biases towards certain amplicons over others (e.g., as in amplification bias). Factors that affect amplification bias include primer affinity to templates, template length, template abundance, and GC-content, as well as other sample and processing reagent characteristics. Such factors lead to unequal sequencing read distributions across different DNA templates within the same sample, which obscure results returned by such processes.

### SUMMARY

Beyond amplification bias, PCR can also produce artifacts such as chimeric molecules and heteroduplex molecules. PCR-resultant chimeric sequences can represent a large percentage of libraries in an undesired manner, which can severely skew diversity analyses for certain sample types (e.g., microbiome samples). In another representative application, tests in precision diagnostics strive to detect extremely small allelic fractions (e.g., ˜0.1% or lower percentages of allelic fractions); however, PCR-resultant artifacts produced during library preparation can result in an unacceptable false-positives or other inaccuracies, thereby requiring more extensive computational filtering and thereby limiting sensitivity for characterization of loci types (e.g., fusion genes). In another specific application, high-throughput immune repertoire sequencing of the VDJ variability, diversity, and joining (VDJ) junction can be significantly affected by PCR artifacts, thereby producing accuracies below 70% while overestimating receptor frequencies by an unacceptable level (e.g., up to 5000-fold). Therefore, PCR-based amplification workflows for next generation sequencing (NGS) require better interventions to reduce false-positive calls.

Multiple methods to limit PCR bias and errors are available with various ranges of complexities, in relation to library preparation operations and post-sequencing computational operations. For instance, selection of low-error polymerases can be used to limit PCR errors during library preparation, and system operators can limit the number of PCR cycles performed for an assay, in order to limit artifact formation. PCR chimeras typically occur more frequently towards later stages of PCR when primer resources are more scarce. However, execution of PCR cycle titration limits may be difficult for workflows that receive heterogeneous sample types, where thermal cycling protocols would need to be optimized for the specific sample type, and cycle limits are not practically feasible. Molecular barcoding methods, such as methods implementing unique molecular identifiers (UMI), are commonly used for post-sequencing correction; however, some drawbacks of implementing UMIs include unequal representation of amplicons in sequencing data and introduction of false UMIs can be during library preparation. While solvable, these drawbacks often require deeper sequencing coverage in order to achieve proper amplicon representation as well as a sufficient level of UMI sampling to remove false-positive barcodes. As such, existing technologies suffer from high degree of complexity, computational expense, execution difficulties associated with various sample types, unequal representation of amplicons, and required use of specialized equipment.

As such, there is a need for innovation in fields relating to library preparation technologies.

Current technologies for library preparation suffer from a high degree of complexity, inherent issues associated with amplification bias and amplification artifact production, sample preparation and processing workflow limitations, high computational burden to correct errors, and required use of specialized equipment, all of which reduce accuracy of returned results.

Accordingly, this disclosure describes embodiments, variations, and examples of systems, methods, and compositions for performing library preparation, which are significantly less subject to or otherwise eliminate issues attributed to bulk amplification approaches for library preparation, as described above.

Library preparation according to the disclosure can additionally or alternatively involve functionalized particles with template nucleic acid molecules coupled thereto and distributed across partitions. Functionalized particles can be magnetic or non-magnetic. Functionalized particles can be porous or non-porous. Functionalized particles can be buoyant or have suitable density properties. Functionalized particles can be configured to swell (e.g., as in a hydrogel within an aqueous solution), or can be configured to not swell in certain environments. Functionalized particles can be configured to controllably degrade in certain environmental conditions.

An aspect of the disclosure provides compositions, methods, and systems for accurate library preparation from a sample distributed across a large number of partitions, with significant performance improvements in relation to: reduction of amplification bias (e.g., PCR bias), reduction of amplification artifact production (e.g., chimeras and other undesired structures), increased efficiency of sample preparation, improvements in partitioning rates, improvements in level of partitioning achievable, reduction of high computational burden to correct errors, and other factors. Such achievements are made without limitations to number of amplification cycles implemented and use of specific materials for PCR (e.g., specific polymerases, materials designed for non-competitive amplification, etc.). Processing materials co-delivered with sample materials across partitions can include limiting reagents (e.g., dNTPs, primers, etc.) that promote generation of a limited amount of product per partition during amplification, in order to further reduce amplification bias effects.

In variations, the disclosure can provide methods, systems, and compositions for accurate library preparation without amplification bias, even when greater than 15 amplification cycles, greater than 16 amplification cycles, greater than 17 amplification cycles, greater than 18 amplification cycles, greater than 19 amplification cycles, greater than 20 amplification cycles, greater than 25 amplification cycles, greater than 30 amplification cycles, greater than 35 amplification cycles, greater than 40 amplification cycles, or greater numbers of amplification cycles (e.g., amplification cycles of a PCR protocol) are involved.

In particular, the disclosure provides methods, systems, and compositions for reducing amplification bias by a factor of 10-fold, 50-fold, 100-fold, 500-fold, 1000-fold, 5000-fold, 10000-fold, or greater, in comparison to current technologies for library preparation involving amplification without partitioning. In particular, due to the large number of partitions involved (where partition numbers achievable are described in more detail below) and low occupancy of partitions by targets (where occupancy characteristics achievable are described in more detail below), the potential for amplification bias for different targets is reduced or entirely eliminated with use of the invention(s) described, as attributed to reduced or eliminated competition during amplification reactions within individual partitions. As a further benefit, amplification bias is reduced or eliminated with use of the invention(s) described, without modification of the number of amplification cycles involved, and/or for samples involving high GC and/or AT content.

As such, given the low level or outright elimination of amplification bias attributed to the invention(s) described, the disclosure provides methods where amplification bias-associated computational corrections are not necessary, thereby providing computational performance enhancements to systems involved in library preparation. In particular, the invention(s) obviate the need for correction methods, such as those involving computational binning of fragment counts and GC/AT counts, estimation of predicted counts for each bin, normalization using Poisson model distributions, and other factors. When performing such correction methods, however, the level of amplification bias determined for the invention(s) described would have amplification bias reduced by factors described above.

The disclosure also provides methods, systems, and compositions for reducing chimera and other artifact production during library preparation, such that artifacts/chimeras represent less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library.

The disclosure also provides methods, systems, and compositions for reducing false positives during library preparation, such that false positives are reduced by a factor of 10-fold, 50-fold, 100-fold, 500-fold, 1000-fold, 5000-fold, 10000-fold, or greater, in comparison to current technologies for library preparation involving amplification.

In example applications of use, the disclosure provides methods for library preparation with orders of magnitudes improvements in performance, for applications where current technologies produce a high level of amplification error/bias, a high false positive rate, or other issues. In one Example, the invention(s) can be applied to immune repertoire sequencing library preparation involving variable, diversity, and joining (VDJ) genes, where potential for high false positives and high PCR error are inherent when standard technologies involving conventional PCR are used. In another example, the invention(s) can be applied to sequencing and library preparation for ribosomal RNAs (e.g., 16S rRNAs, ITS rRNAs, etc.) of a sample, where potential for high false positives and high PCR error are inherent when standard technologies involving conventional PCR are used. In another example, the invention(s) can be applied to sequencing and library preparation for precision diagnostics, where potential for high false positives and high PCR error are inherent when standard technologies involving conventional PCR are used. In another example, the invention(s) can be applied to whole genome sequencing cases (e.g., single cell whole genome sequencing), where high levels of amplification bias are inherent when standard technologies involving conventional PCR are used.

In relation to immune repertoire sequencing of the VDJ junction (VDJ-seq), the disclosure provides methods, systems, and compositions that can be used to reduce or eliminate PCR bias and chimera/artifact generation during sample processing, to achieve greater than 70% accuracy, greater than 80% accuracy, greater than 90% accuracy, greater than 91% accuracy, greater than 92% accuracy, greater than 93% accuracy, greater than 94% accuracy, greater than 95% accuracy, greater than 96% accuracy, greater than 97% accuracy, greater than 98% accuracy, or greater than 99% accuracy in VDJ-seq results. Furthermore, in relation to immune repertoire sequencing of the VDJ junction (VDJ-seq), the disclosure provides methods, systems, and compositions that are not subject to overestimations of receptor frequencies, where standard technologies inherently overestimate receptor frequency by up to 5000-fold.

In relation to rRNA applications, the disclosure provides methods, systems, and compositions that reduce chimera and other artifact production during library preparation, such that artifacts/chimeras represent less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library, thereby improving accuracy of analyses of diversity (e.g., microbiome diversity) within a sample. An example use case involves analyses of samples involving hypervariable regions of rRNA (e.g., v3-v5 regions of 16S rRNA). In particular, accurate library preparation for microbiome samples results in improved accuracy of diversity analyses, abundance inferences, and other important metrics for characterizations.

In relation to applications in precision diagnostics, the disclosure provides methods, systems, and compositions that are capable of accurate library preparation for samples involving extremely small allelic fractions, such as allelic fractions less than 1%, less than 0.9%, less than 0.8%, less than 0.7%, less than 0.6%, less than 0.4%, less than 0.3%, less than 0.2%, less than 0.1%, less than 0.05%, or less.

Applications of use include library preparation for detection of oncogenic gene fusions (e.g., anaplastic lymphoma kinase (ALK) fusions, BCR-ABL fusions, etc.). Accuracy is enhanced due to aspects of the invention(s) that result in artifacts/chimeras representing less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library, as well as reductions in false-positive rates by a factor of 10-fold, 50-fold, 100-fold, 500-fold, 1000-fold, 5000-fold, 10000-fold, or greater. In particular, accurate library preparation for samples results in improved accuracy of, abundance inferences associated with copy number variations, and other important metrics for characterizations.

In relation to sample partitioning and processing, the disclosure provides methods, systems, and compositions for generating an extremely high number of droplets (e.g., greater than 200,000 droplets, greater than 300,000 droplets, greater than 400,000 droplets, greater than 500,000 droplets, greater than 1 million droplets, greater than 2 million droplets, greater than 3 million droplets, greater than 4 million droplets, greater than 5 million droplets, greater than 6 million droplets, greater than 7 million droplets, greater than 8 million droplets, greater than 9 million droplets, greater than 10 million droplets, greater than 15 million droplets, greater than 20 million droplets, greater than 25 million droplets, greater than 30 million droplets, greater than 40 million droplets, greater than 50 million droplets, greater than 100 million droplets, etc.) within a collecting container having a volumetric capacity (e.g., less than 50 microliters, from 50 through 100 microliters and greater, etc.), where droplets have a characteristic dimension (e.g., from 1-50 micrometers, from 10-70 micrometers, etc.) that is relevant for digital analyses, target detection, individual molecule partitioning, or other applications. In applications, characteristic dimensions of droplets are approximately 10 micrometers, 20 micrometers, 30 micrometers, 40 micrometers, 50 micrometers, 60 micrometers, 70 micrometers, or 80 micrometers, where the droplets are highly monodisperse and have a coefficient of variation less than 20%, 15%, 10%, 5%, 4%, 3%, 2% or less, in relation to droplet morphologies.

In relation to occupancy, the disclosure provides methods, systems, and compositions for sample partitioning, where each partition has one or zero molecules, such that the partitions are characterized as having low occupancy (e.g., less than 40% occupancy of partitions by individual molecules, less than 30% occupancy of partitions by individual molecules, less than 20% occupancy of partitions by individual molecules, less than 19% occupancy of partitions by individual molecules, less than 18% occupancy of partitions by individual molecules, less than 17% occupancy of partitions by individual molecules, less than 16% occupancy of partitions by individual molecules, less than 15% occupancy of partitions by individual molecules, less than 14% occupancy of partitions by individual molecules, less than 13% occupancy of partitions by individual molecules, less than 12% occupancy of partitions by individual molecules, less than 11% occupancy of partitions by individual molecules, less than 10% occupancy of partitions by individual molecules, less than 9% occupancy of partitions by individual molecules, less than 8% occupancy of partitions by individual molecules, less than 7% occupancy of partitions by individual molecules, less than 6% occupancy of partitions by individual molecules, less than 5% occupancy of partitions by individual molecules, less than 4% occupancy of partitions by individual molecules, less than 3% occupancy of partitions by individual molecules, less than 2% occupancy of partitions by individual molecules, etc.).

In relation to occupancy, partitions/droplets can be characterized by a lambda value (e.g., a value characterizing mean number of molecules/targets per droplet) less than or equal to 3, less than or equal to 2, less than or equal to 1.5, less than or equal to 1, less than or equal to 0.5, less than or equal to 0.25, etc.

In example applications involving cell-free DNA (cfDNA) extraction and analysis, the disclosure provides methods, systems, and compositions for partitioning cfDNA molecules in a low occupancy manner, as described above, thereby allowing for single molecule amplification without competition, within most partitions. In one example use case, a majority of ultra-partitioned library preparation droplets produced according to methods described have ≤2 of cDNA molecules for unique amplification, resulting in significant reductions in PCR error. The example use case involved with a 10,000 cell input for cfDNA extraction, with 100 μL of PCR reagent used.

In examples, the systems, methods, and compositions described can be used to generate 1,000 counts per target for each of a set of targets of interest, 2,000 counts per target for each of a set of targets of interest, 3,000 counts per target for each of a set of targets of interest, 4,000 counts per target for each of a set of targets of interest, 5,000 counts per target for each of a set of targets of interest, 6,000 counts per target for each of a set of targets of interest, 7,000 counts per target for each of a set of targets of interest, 8,000 counts per target for each of a set of targets of interest, 9,000 counts per target for each of a set of targets of interest, 10,000 counts per target for each of a set of targets of interest, 20,000 counts per target for each of a set of targets of interest, 30,000 counts per target for each of a set of targets of interest, 40,000 counts per target for each of a set of targets of interest, 50,000 counts per target for each of a set of targets of interest, 60,000 counts per target for each of a set of targets of interest, 70,000 counts per target for each of a set of targets of interest, 80,000 counts per target for each of a set of targets of interest, 90,000 counts per target for each of a set of targets of interest, 100,000 counts per target for each of a set of targets of interest, 120,000 counts per target for each of a set of targets of interest, 130,000 counts per target for each of a set of targets of interest, 140,000 counts per target for each of a set of targets of interest, 150,000 counts per target for each of a set of targets of interest, 160,000 counts per target for each of a set of targets of interest, 170,000 counts per target for each of a set of targets of interest, 180,000 counts per target for each of a set of targets of interest, 190,000 counts per target for each of a set of targets of interest, 200,000 counts per target for each of a set of targets of interest, 210,000 counts per target for each of a set of targets of interest, 220,000 counts per target for each of a set of targets of interest, 230,000 counts per target for each of a set of targets of interest, 240,000 counts per target for each of a set of targets of interest, 250,000 counts per target for each of a set of targets of interest, 260,000 counts per target for each of a set of targets of interest, 270,000 counts per target for each of a set of targets of interest, 280,000 counts per target for each of a set of targets of interest, 290,000 counts per target for each of a set of targets of interest, 300,000 counts per target for each of a set of targets of interest, or other counts per target for each of a set of targets of interest.

The disclosure provides compositions, methods, and systems for multiplexed target processing for library preparation, where compositions can be configured for 20-plex amplification of loci of interest for each a set of targets being analyzed, 30-plex amplification of loci of interest for each a set of targets being analyzed, 40-plex amplification of loci of interest for each a set of targets being analyzed, 50-plex amplification of loci of interest for each a set of targets being analyzed, 60-plex amplification of loci of interest for each a set of targets being analyzed, 70-plex amplification of loci of interest for each a set of targets being analyzed, 80-plex amplification of loci of interest for each a set of targets being analyzed, 90-plex amplification of loci of interest for each a set of targets being analyzed, 100-plex amplification of loci of interest for each a set of targets being analyzed, or greater.

Relatedly, an aspect of the disclosure provides embodiments, variations, and examples of devices and methods for rapidly generating partitions (e.g., droplets from a sample fluid, droplets of an emulsion) and distributing nucleic acid material of a sample across partitions, where, the device includes: a first substrate defining a reservoir comprising a reservoir inlet and a reservoir outlet; a membrane coupled to the reservoir outlet and comprising a distribution of holes; and a supporting body comprising an opening configured to retain a collecting container in alignment with the reservoir outlet. During operation, the first substrate can be coupled with the supporting body and enclose the collecting container, with the reservoir outlet aligned with and/or seated within the collecting container. During operation, the reservoir can contain a sample fluid (e.g., a mixture of nucleic acids of the sample and materials for an amplification reaction), where application of a force to the device or sample fluid generates a plurality of droplets within the collecting container at an extremely high rate (e.g., of at least 200,000 droplets/minute, of at least 300,000 droplets/minute, of at least 400, droplets/minute, of at least 500,000 droplets/minute, of at least 600,000 droplets/minute, of at least 700,000 droplets/minute, of at least 800,000 droplets/minute, of at least 900,000 droplets/minute, of at least 1 million droplets/minute, of at least 2 million droplets/minute, of at least 3 million droplets/minute, of at least 4 million droplets/minute, of at least 5 million droplets/minute, of at least 6 million droplets per minute, etc.), where the droplets are stabilized in position (e.g., in a close-packed format, in equilibrium stationary positions) within the collecting container.

An aspect of the disclosure provides embodiments, variations, and examples of a method for rapidly generating partitions (e.g., droplets from a sample fluid, droplets of an emulsion) within a collecting container at an extremely high rate, each of the plurality of droplets including an aqueous mixture for a digital analysis, wherein upon generation, the plurality of droplets is stabilized in position (e.g., in a close-packed format, at equilibrium stationary positions, etc.) within a continuous phase (e.g., as an emulsion having a bulk morphology defined by the collecting container). In aspects, partition generation can be executed by driving the sample fluid through a distribution of holes of a membrane (e.g., driving the sample through a membrane comprising a distribution of holes, the membrane coupled to a reservoir outlet of a reservoir for the sample, and the reservoir aligned with the collecting container), where the applied force can be one or more of centrifugal (e.g., under centrifugal force), associated with applied pressure, magnetic, or otherwise physically applied. As such, driving the sample can include spinning the sample within the reservoir, the membrane, and the collecting container within a centrifuge.

In relation to a single-tube workflow in which the collecting container remains closed (e.g., the collecting container has no outlet, there is no flow out of the collecting container, to avoid sample contamination), method(s) can further include transmitting heat to and from the plurality of droplets within the closed collecting container according to an assay protocol. In relation to generation of emulsions having suitable clarity (e.g., with or without refractive index matching), method(s) can further include transmission of signals from individual droplets from within the closed collecting container, for readout (e.g., by an optical detection platform, by another suitable detection platform).

The disclosure also provides compositions that produce significantly improved signal-to-noise (SNR) values with reduced background, in relation to detection techniques described below (e.g., based on lightsheet imaging, etc.) for partitions arranged in bulk in 3D. In examples, target signals can be at least 10² greater than background noise signals, 10³ greater than background noise signals, 10⁴ greater than background noise signals, 10⁵ greater than background noise signals, 10⁶ greater than background noise signals, 10⁷ greater than background noise signals, or better. Background noise can be attributed to fluorescence from adjacent partitions and adjacent planes of the set of planes of partitions in the context of emulsion digital PCR, or attributed to other sources with closely-positioned partitions.

In examples associated with reaction materials described and used for droplet digital PCR, determining the target signal value can include: for each plane of a set of planes of partitions under interrogation (e.g., by lightsheet detection, by another method of detection, etc.): determining a categorization based upon a profile of positive partitions represented in a respective plane, determining a target signal distribution and a noise signal distribution specific to the profile, and determining a target signal intensity and a noise signal intensity for the respective plane. Here, the target signal value can be an average value (or other representative value) of the target signal intensities determined from the set of planes, and the background noise signal value can be an average value (or other representative value) of the noise signal intensities determined from the set of planes.

Where method(s) include transmitting heat to and from the plurality of droplets, within the closed container, the droplets are stable across a wide range of temperatures (e.g., 1° C. through 95° C., greater than 95° C., less than 1° C.) relevant to various digital analyses and other bioassays, where the droplets remain consistent in morphology and remain unmerged with adjacent droplets.

The disclosure generally provides mechanisms for efficient capture, distribution, and labeling of target material (e.g., DNA, RNA, miRNA, proteins, small molecules, single analytes, multianalytes, etc.) in order to enable characterization of materials, in parallel and in a multiplexed manner, for various applications.

In examples, the approach discussed is designed around a simple workflow to enable deployment to local and decentralized laboratories. First, samples are carried end-to-end in the same PCR tube for user convenience and to minimize sample contamination. Second, ultra-partitioning and amplification can be performed in standard laboratory equipment such as a swing bucket centrifuge and thermal cycler, lowering the infrastructure cost in comparison to other library preparation and sequencing platforms.

Another aspect of the present disclosure provides a non-transitory computer readable medium comprising machine executable code that, upon execution by one or more computer processors, implements any of the methods above or elsewhere herein.

The disclosure provides compositions, methods, and systems for multiplexed detection of targets that can provide value in research or other non-clinical settings, with or without evaluation and processing of live human or mammalian biological material, and without the immediate purpose of obtaining a diagnostic result of a disease or health condition.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. The present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference in their entireties for all purposes and to the same extent as if each individual publication, patent, or patent application was specifically and individually indicated to be incorporated by reference.

Furthermore, where a range of values is provided, it is understood that each intervening value, between the upper and lower limit of that range and any other stated or intervening value in that stated range is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either both of those included limits are also included in the invention.

### BRIEF DESCRIPTION OF THE FIGURES

FIG. 1 depicts a flowchart and schematic of an embodiment of a method for library preparation.

FIG. 2 depicts a schematic of an embodiment of a system for partitioning samples for library preparation applications.

FIG. 3A depicts a schematic of an example method for single cell whole genome library preparation.

FIG. 3B depicts data depicting an estimated % of partitions with ≤1 or ≤2 copies of cDNA transcripts in a single cell RNA-sequencing sample, where, with a 10,000 cell input, almost all library preparation partitions have ≤2 cDNA molecules derived from single cell material for unique amplification.

FIG. 4 depicts a schematic of an example method for 16s rRNA library preparation.

FIG. 5 depicts outputs of library preparation processes using a dropletized partition workflow in comparison to a ‘bulk’ amplification workflow.

FIG. 6 illustrates a computer system that is programmed or otherwise configured to implement methods provided herein.

### DETAILED DESCRIPTION OF THE INVENTION(S)

While various embodiments of the invention have been shown and described herein, it will be obvious to those skilled in the art that such embodiments are provided by way of example only. Numerous variations, changes, and substitutions can occur to those skilled in the art without departing from the invention. It should be understood that various alternatives to the embodiments of the invention described herein can be employed.

### 1. General Overview

The invention(s) described can confer several benefits over conventional systems, methods, and compositions for library preparation. In particular, the invention(s) described cover embodiments, variations, and examples of systems, methods, and compositions for library preparation, which are significantly less subject to or otherwise eliminate issues attributed to bulk amplification approaches. In more detail, the invention(s) achieve significantly improved levels of accurate library preparation performance, for a sample distributed across a large number of partitions, with significant improvements in relation to: reduction of amplification bias (e.g., PCR bias), reduction of amplification artifact production (e.g., chimeras and other undesired structures), increased efficiency of sample preparation, improvements in partitioning rates, improvements in level of partitioning achievable, reduction of high computational burden to correct errors, and other factors. Such achievements are made without limitations to number of amplification cycles implemented and use of specific materials for PCR (e.g., specific polymerases, materials designed for non-competitive amplification, etc.).

In particular, the disclosure provides methods, systems, and compositions for reducing amplification bias by a factor of 10-fold, 50-fold, 100-fold, 500-fold, 1000-fold, 5000-fold, 10000-fold, or greater, in comparison to current technologies for library preparation involving amplification without partitioning. The methods, systems, and compositions thus preserve the true composition and relative representation of species/targets/etc. of an original sample (e.g., input sample). Given the low level or outright elimination of amplification bias attributed to the invention(s) described, the disclosure provides methods where amplification bias-associated computational corrections are not necessary, thereby providing computational performance enhancements to systems involved in library preparation.

The invention(s) also reduce chimera and other artifact production during processing operations for library preparation, such that artifacts/chimeras represent less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library. The invention(s) also reduce false positive sequences observed during library preparation, by a factor of 10-fold, 50-fold, 100-fold, 500-fold, 1000-fold, 5000-fold, 10000-fold, or greater, in comparison to current technologies for library preparation involving amplification.

The inventions achieve unprecedented performance in comparison to current technologies that produce a high level of amplification error/bias, a high false positive rate, or other issues. In one example, the invention(s) can be applied to immune repertoire sequencing library preparation involving variable, diversity, and joining (VDJ) genes, where potential for high false positives and high PCR error are inherent when standard technologies involving conventional PCR are used. In another example, the invention(s) can be applied to sequencing and library preparation for ribosomal RNAs (e.g., 16S and/or 18S rRNAs, ITS rRNAs, etc.) of a sample, where potential for high false positives and high PCR error are inherent when standard technologies involving conventional PCR are used. In another example, the invention(s) can be applied to sequencing and library preparation for precision diagnostics, where potential for high amplification bias, high false positives and high PCR error are inherent when standard technologies involving conventional PCR are used. In another example, the invention(s) can be applied to whole genome sequencing cases (e.g., single cell whole genome sequencing), where high levels of amplification bias are inherent when standard technologies involving conventional PCR are used.

In relation to sample partitioning and processing, the invention(s) generate an extremely high number of droplets (e.g., greater than 200,000 droplets, greater than 300,000 droplets, greater than 400,000 droplets, greater than 500,000 droplets, greater than 1 million droplets, greater than 2 million droplets, greater than 3 million droplets, greater than 4 million droplets, greater than 5 million droplets, greater than 6 million droplets, greater than 7 million droplets, greater than 8 million droplets, greater than 9 million droplets, greater than 10 million droplets, greater than 15 million droplets, greater than 20 million droplets, greater than 25 million droplets, greater than 30 million droplets, greater than 40 million droplets, greater than 50 million droplets, greater than 100 million droplets, etc.) within a collecting container having a volumetric capacity (e.g., less than 50 microliters, from 50 through 100 microliters and greater, etc.), where droplets have a characteristic dimension (e.g., from 1-50 micrometers, from 10-70 micrometers, etc.) that is relevant for digital analyses, target detection, individual molecule partitioning, or other applications. In applications, characteristic dimensions of droplets are approximately 10 micrometers, 20 micrometers, 30 micrometers, 40 micrometers, 50 micrometers, 60 micrometers, 70 micrometers, or 80 micrometers, where the droplets are highly monodisperse and have a coefficient of variation less than 20%, 15%, 10%, 5%, 4%, 3%, 2% or less, in relation to droplet morphologies.

In relation to occupancy, the invention(s) achieve rapid partitioning, where each partition has one or zero molecules, such that the partitions are characterized as having low occupancy (e.g., less than 40% occupancy of partitions by individual molecules, less than 30% occupancy of partitions by individual molecules, less than 20% occupancy of partitions by individual molecules, less than 19% occupancy of partitions by individual molecules, less than 18% occupancy of partitions by individual molecules, less than 17% occupancy of partitions by individual molecules, less than 16% occupancy of partitions by individual molecules, less than 15% occupancy of partitions by individual molecules, less than 14% occupancy of partitions by individual molecules, less than 13% occupancy of partitions by individual molecules, less than 12% occupancy of partitions by individual molecules, less than 11% occupancy of partitions by individual molecules, less than 10% occupancy of partitions by individual molecules, less than 9% occupancy of partitions by individual molecules, less than 8% occupancy of partitions by individual molecules, less than 7% occupancy of partitions by individual molecules, less than 6% occupancy of partitions by individual molecules, less than 5% occupancy of partitions by individual molecules, less than 4% occupancy of partitions by individual molecules, less than 3% occupancy of partitions by individual molecules, less than 2% occupancy of partitions by individual molecules, etc.). As such, Poisson error corrections due to partitioning bias are not necessary for accurate results.

The disclosure provides methods, systems, and compositions for partitioning and processing other samples with low starting input material (e.g., in volume, in number of targets contained within starting input material, etc.), where such samples are prone to overamplification and therefore, amplification bias and chimera formation.

In example applications involving cell-free DNA (cfDNA) extraction and analysis, the disclosure provides methods, systems, and compositions for partitioning cfDNA molecules in a low occupancy manner, as described above, thereby allowing for single molecule amplification without competition, within most partitions. In one example use case, a majority of ultra-partitioned library preparation droplets produced according to methods described have ≤2 of cDNA molecules for unique amplification, resulting in significant reductions in PCR/amplification bias/allelic bias or dropout, preserving true representation of copy number variation (CNV) and single nucleoetide polymorphism (SNP) statuses of the original sample. The example use case involved with a 10,000 cell input for cfDNA extraction, with 100 μL of PCR reagent used.

The disclosure provides methods, systems, and compositions for accurately generating and amplifying single cell RNA-seq libraries, in order to preserve the original representation of the library (e.g., the original representation of distributions present in the original input sample) and reducing the amount of sequencing required in order to discover all molecules present. In particular, some molecules of an input sample may be amplified more efficiently than others so compared to methods as described herein, other methods may require more significant sequencing efforts in order to discover those molecules that are amplified less relative to other molecules of the input sample.

The inventions provide a platform for extremely stable emulsion formulation. Generated emulsions for library preparation and digital analyses are stable across a wide range of temperatures (e.g., in relation to temperatures involved in thermal cycling, in relation to cold storage post-sample processing, etc.). Furthermore, in representative examples, droplets/partitions generated using embodiments of the systems described have a higher emulsification rate than other emulsification technologies, and demonstrate lower sample loss (e.g., greater than 97% combined efficiency) compared to microfluidic droplet workflows that can have greater than 30% dead volume. As such, variations of the invention(s) described can achieve dead volumes (i.e., amount of sample that is not partitioned for further analyses) less than 10%, less than 9%, less than 8%, less than 7%, less than 6%, less than 5%, less than 4%, less than 3%, or lower levels.

The invention(s) can be applied to samples from human organisms, other multicellular animals, plants, fungi, unicellular organisms, viruses, and/or other material, with respect to accurate library preparation. Downstream characterizations of targets involved can then be used for diagnostic purposes and/or for generation of targeted therapies to improve states of organisms from which the samples were sourced. The invention(s) can also provide value in research or other non-clinical settings, with or without evaluation and processing of live human or mammalian biological material, and without the immediate purpose of obtaining a diagnostic result of a disease or health condition.

The invention(s) also confer(s) the benefit of providing mechanisms for efficient capture and labeling of target material (e.g., DNA, RNA, miRNA, proteins, small molecules, single analytes, multianalytes, etc.) in order to enable genomic, proteomic, and/or other multi-omic characterization of materials for various applications.

Additionally or alternatively, the invention(s) can confer any other suitable benefit.

### 2. Methods and Materials

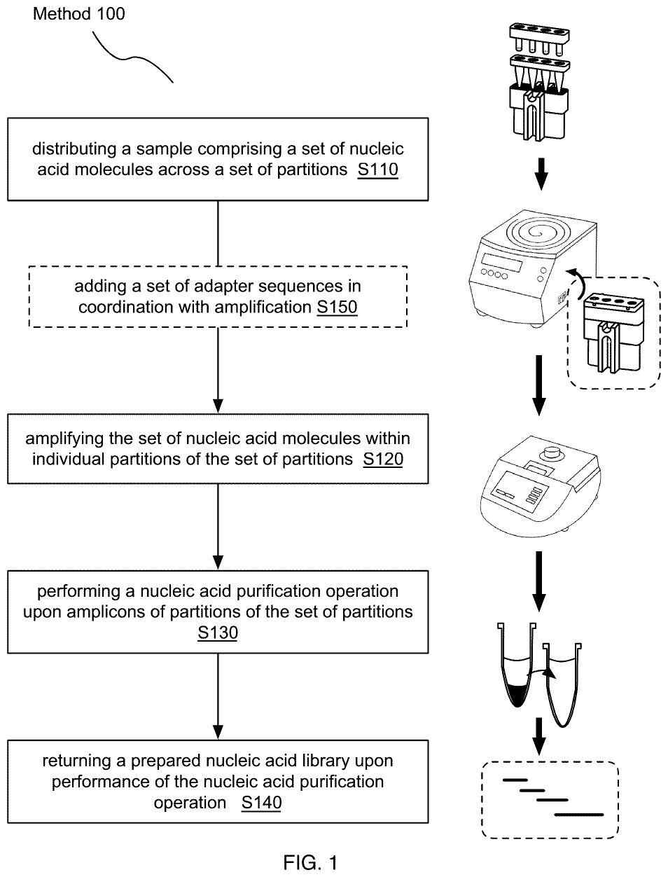

As shown in FIG. 1, embodiments of a method 100 for library preparation include: distributing a sample comprising a set of nucleic acid molecules across a set of partitions at low occupancy (e.g., at levels of occupancy described), such that a partition of the set of partitions in a representative state contains a single nucleic acid molecule of the set of nucleic acid molecules S110; amplifying the set of nucleic acid molecules within individual partitions of the set of partitions for a set of amplification cycles, such that the partition in the representative state contains a set of amplicons generated from the single nucleic acid molecule S120; performing a nucleic acid purification operation upon amplicons of partitions of the set of partitions S130; and returning a prepared nucleic acid library upon performance of the nucleic acid purification operation S140, where the prepared nucleic acid library comprises an amplification bias below a first threshold level and a percentage of chimeric sequences below a second threshold level. In variations, the method 100 can further include adding a set of adapter sequences in coordination with amplification, the set of adapter sequences corresponding to a sequencing platform S150. As such, the method 100 functions to generate highly accurate libraries from a sample of nucleic acids, without involving limitations to the number of amplification cycles used and/or specific materials (e.g., polymerases) to reduce amplification bias, false positive rates, and generated artifact sequences.

Prepared nucleic acid libraries generated according to methods described have accuracy characteristics, levels of chimeric sequence representation in final libraries, false positive rates, reduced or eliminated levels of amplification bias, and other characteristics, as described in more detail.

In particular, embodiments of the method 100 are capable of achieving high levels of accuracy, in comparison to current technologies for library preparation involving amplification without partitioning. In particular, due to the large number of partitions involved and low occupancy of partitions by targets, the potential for amplification bias for different targets is reduced or entirely eliminated when performing embodiments of the method 100 described, as attributed to reduced or eliminated competition during amplification reactions within individual partitions. As a further benefit, amplification bias is reduced or eliminated, without modification of the number of amplification cycles involved, without use of materials (e.g., polymerases, etc.) specifically designed to be less subject to amplification bias, and/or for samples involving high GC and/or AT content.

Embodiments of the method 100 can be performed by embodiments, variations, and examples of system components described in U.S. application Ser. No. 17/230,907 filed on 14 Apr. 2021 and/or U.S. application Ser. No. 17/687,080 filed 4 Mar. 2022, which are each hereby incorporated in its entirety by this reference.

### 2.1 Method-Sample Types and Targets

In variations, the method 100 can be used to process sample types including biological fluids including or derived from one or more of: blood (e.g., whole blood, peripheral blood, non-peripheral blood, blood lysate, etc.), saliva, reproductive fluids, mucus, pleural fluid, pericardial fluid, peritoneal fluid, amniotic fluids, otic fluid, sweat, interstitial fluid, synovial fluid, cerebral-spinal fluid, urine, gastric fluids, biological waste, other biological fluids; tissues (e.g., homogenized tissue samples); food samples; liquid consumable samples; and/or other sample materials. Samples can be derived from human organisms, other multicellular animals, plants, fungi, unicellular organisms, viruses, and/or other material. In specific examples, samples processed can include maternal samples (e.g., blood, plasma, serum, urine, chorionic villus, etc.) including maternal and fetal material (e.g., cellular material, cell-free nucleic acid material, other nucleic acid material, etc.) from which prenatal detection or diagnosis of genetic disorders (e.g., aneuploidies, genetically inherited diseases, other chromosomal issues, etc.) can be performed. Samples processed can include samples associated with cancerous tissue (e.g., tissue-derived samples, samples carrying circulating tumor cells, other samples), samples from which immune responses of a subject can be determined, samples associated with pathogen detection, microbiome samples (e.g., associated with agriculture, associated with food production, associated with viticulture, associated with other consumables, taken from a mammalian subject, taken from a non-mammalian subject, taken from the environment, etc.), plasmid samples, cell samples from which cfDNA can be extracted, and/or other sample types.

In embodiments, libraries prepared according to embodiments, variations, and examples of the method 100 can include libraries derived from or otherwise associated with nucleic acids, including DNA, cDNA, genomic DNA, nucleosomal DNA, RNA, mRNA, miRNA, or other nucleic acids.

### 2.2 Method-Sample Partitioning

Step S110 recites: distributing a sample comprising a set of nucleic acid molecules across a set of partitions at low occupancy, such that a partition of the set of partitions in a representative state contains a single nucleic acid molecule of the set of nucleic acid molecules. Sample distribution operations described function to partition nucleic acid molecules across a large number of partitions and at low occupancy, in order to reduce or eliminate potentials for amplification bias, amplification artifact generation, false positive rates, and other issues typically associated with library preparation for various sample types.

In relation to low occupancy of the set of partitions associated with operation S110, system components described distribute the input sample across partitions, such that each partition is occupied by less than or equal to one molecule, or alternatively, such that each partition is occupied by less than or equal to two molecules (i.e., low occupancy). In variations, the percent of non-empty partitions (i.e., partitions/droplets containing at least one nucleic acid molecule) having less than or equal to 1 molecule can be: greater than 70%, greater than 75%, greater than 80%, greater than 85%, greater than 90%, or greater. In variations, the percent of non-empty partitions (i.e., partitions/droplets containing at least one nucleic acid molecule) having less than or equal to 2 molecules can be: greater than 70%, greater than 75%, greater than 80%, greater than 85%, greater than 90%, greater than 91%, greater than 92%, greater than 93%, greater than 94%, greater than 95%, greater than 96%, greater than 97%, or greater. The percentages described are dependent upon input sample characteristics and numbers of partitions generated using partition systems (an embodiment of which is described below).

In relation to low occupancy of the set of partitions associated with operation S110, non-empty partitions can represent greater than 25% of the total number of partitions generated from an input sample, greater than 30% of the total number of partitions generated from an input sample, greater than 35% of the total number of partitions generated from an input sample, greater than 40% of the total number of partitions generated from an input sample, greater than 45% of the total number of partitions generated from an input sample, greater than 50% of the total number of partitions generated from an input sample, greater than 55% of the total number of partitions generated from an input sample, greater than 60% of the total number of partitions generated from an input sample, greater than 65% of the total number of partitions generated from an input sample, greater than 70% of the total number of partitions generated from an input sample, greater than 75% of the total number of partitions generated from an input sample, or greater than 80% of the total number of partitions generated from an input sample.

Alternatively, in relation to low occupancy of the set of partitions associated with operation S110, at least 10% of the set of partitions can contain no nucleic acid molecules from the input sample, at least 20% of the set of partitions can contain no nucleic acid molecules from the input sample, at least 40% of the set of partitions can contain no nucleic acid molecules from the input sample, at least 50% of the set of partitions can contain no nucleic acid molecules from the input sample, at least 60% of the set of partitions can contain no nucleic acid molecules from the input sample, at least 70% of the set of partitions can contain no nucleic acid molecules from the input sample, at least 80% of the set of partitions can contain no nucleic acid molecules from the input sample, or greater percentages of the set of partitions can contain no nucleic acid molecules from the input sample.

As such, a percentage of partitions of the set of partitions in the representative state can be greater than 70%, greater than 75%, greater than 80%, greater than 85%, greater than 90%, or a greater percentage of non-empty partitions.

In relation to operation S110, the set of partitions includes droplets of an emulsion generated using system components described in more detail below, where droplets each have a characteristic dimension (e.g., diameter) less than 70 micrometers, less than 65 micrometers, less than 60 micrometers, less than 55 micrometers, less than 54 micrometers, less than 53 micrometers, less than 52 micrometers, less than 51 micrometers, less than 50 micrometers, less than 49 micrometers, less than 48 micrometers, less than 47 micrometers, less than 46 micrometers, less than 45 micrometers, less than 44 micrometers, less than 43 micrometers, less than 42 micrometers, less than 41 micrometers, less than 40 micrometers, less than 39 micrometers, less than 38 micrometers, less than 37 micrometers, less than 36 micrometers, less than 35 micrometers, less than 34 micrometers, less than 33 micrometers, less than 32 micrometers, less than 31 micrometers, less than 30 micrometers, less than 29 micrometers, less than 28 micrometers, less than 27 micrometers, less than 26 micrometers, less than 25 micrometers, less than 24 micrometers, less than 23 micrometers, less than 22 micrometers, less than 21 micrometers, less than 20 micrometers, less than 15 micrometers, or less.

In relation to operation S110, generated droplets are additionally characterized as having a low degree of polydispersity (e.g., less than 15% coefficient of variation for polydispersity, less than 14% coefficient of variation for polydispersity, less than 13% coefficient of variation for polydispersity, less than 12% coefficient of variation for polydispersity, less than 11% coefficient of variation for polydispersity, less than 10% coefficient of variation for polydispersity, less than 9% coefficient of variation for polydispersity, less than 8% coefficient of variation for polydispersity, less than 7% coefficient of variation for polydispersity, less than 6% coefficient of variation for polydispersity, less than 5% coefficient of variation for polydispersity, etc.).

In relation to operation S110, the set of partitions can include: greater than 200,000 droplets, greater than 300,000 droplets, greater than 400,000 droplets, greater than 500,000 droplets, greater than 1 million droplets, greater than 2 million droplets, greater than 3 million droplets, greater than 4 million droplets, greater than 5 million droplets, greater than 6 million droplets, greater than 7 million droplets, greater than 8 million droplets, greater than 9 million droplets, greater than 10 million droplets, greater than 15 million droplets, greater than 20 million droplets, greater than 25 million droplets, greater than 30 million droplets, greater than 40 million droplets, greater than 50 million droplets, greater than 100 million droplets, or greater, depending upon input sample size, partitioning system characteristics, and collecting container volume.

In relation to operation S110, embodiments, variations, and examples of system components described can generate a plurality of droplets within the collecting container at an extremely high rate (e.g., of at least 200,000 droplets/minute, of at least 300,000 droplets/minute, of at least 400, droplets/minute, of at least 500,000 droplets/minute, of at least 600,000 droplets/minute, of at least 700,000 droplets/minute, of at least 800,000 droplets/minute, of at least 900,000 droplets/minute, of at least 1 million droplets/minute, of at least 2 million droplets/minute, of at least 3 million droplets/minute, of at least 4 million droplets/minute, of at least 5 million droplets/minute, of at least 6 million droplets per minute, etc.), where the droplets are stabilized in position within a closed collecting container.

When generating droplets in variations of Step S110, input sample material can be combined with materials for the amplification reaction, where the materials can include a master mixture for polymerase chain reaction (PCR) amplification methods or non-PCR-based amplification methods (e.g., multiple displacement amplification (MDA), rolling circle amplification (RCA), strand displacement amplification (SDA), etc.). The master mixture can include a polymerase, dNTPs, buffer components, nuclease free water, primers, and/or other suitable materials. The master mixture can further include limiting reagents (e.g., dNTPs, primers, etc.) that promote generation of a limited amount of product per partition during amplification, in order to further reduce amplification bias effects. The master mixture can additionally or alternatively include components that further prevent artifact sequence (e.g., chimeric sequence) generation or amplification bias, such as components that produce dideoxynucleotides for chain termination, where library intermediates having a dideoxy nucleotide (e.g., at their 3′ end) are unable to serve as primers for further chain extension in subsequent rounds of library generation. As such, generation of chimeric sequences constituents through annealing of a library intermediate to a random or repeated region of a genome and polymerase-directed extension from that region is prevented.

Polymerases can include non high-fidelity polymerases and/or high-fidelity polymerases. Example polymerases for use in PCR operations in connection with the systems, methods, and compositions of the present disclosure can include Taq polymerases (e.g., One Taq® DNA polymerase, Taq DNA polymerase, etc.). Example polymerases for specialty PCR can include: LongAmp® Taq DNA polymerase, Hemo Klen Taq polymerase, Epimark® Hot Start Taq DNA polymerase, and others. Example high-fidelity polymerases for PCR can include: Q5® high-fidelity DNA polymerase, Q5U® hot start high-fidelity DNA polymerase, Phusion® high-fidelity DNA polymerase, and others. Example polymerases for isothermal amplification and strand displacement include: Bst DNA polymerase (full length), Bst DNA polymerase (large fragment), Bst 2.0 DNA polymerase, Bst 3.0 DNA polymerase, Bsu DNA polymerase (large fragment), phi29 DNA polymerase, and others. The master mixture can additionally or alternatively include polymerases for DNA manipulation, such as T7 DNA polymerase, sulfolobus DNA polymerase IV, Therminator™ DNA polymerase, DNA polymerase I (for E. Coli), DNA polymerase I (for large Klenow fragments), T4 DNA polymerase, and others. The master mixture can additionally or alternatively include legacy polymerases, including Vent® (exo-) DNA polymerase, Deep Vent® DNA polymerase, Deep Vent® (exo-) DNA polymerase, and others.

The sample/processing materials can further include functionalized particles with template nucleic acid molecules coupled thereto and distributed across partitions. Functionalized particles can be magnetic or non-magnetic. Functionalized particles can be porous or non-porous. Functionalized particles can be buoyant or have suitable density properties. Functionalized particles can be configured to swell (e.g., as in a hydrogel within an aqueous solution), or can be configured to not swell in certain environments. Functionalized particles can be configured to controllably degrade in certain environmental conditions.

Distributing a sample comprising a set of nucleic acid molecules across a set of partitions in operation S110 can include receiving a sample (variations and examples of which are described above) at a vessel passively or actively (e.g., with applied force, such as with gravitational force, with centrifugal force, with pressurization, etc.). The sample and processing materials can be delivered manually (e.g., with a fluid aspiration and delivery device, such as a pipettor). The sample and processing materials can additionally or alternatively be delivered with automation (e.g., using liquid handling apparatus or other sample handling apparatus).

In variations, vessel formats can include: tubes (e.g., PCR tubes) containing partitions of the sample (e.g., in droplet format, in emulsion format, in another format), wells (e.g., microwells, nanowells, etc.), channels, chambers, and/or other suitable containers. Additionally or alternatively, alternative variations of operation S110 can include receiving the sample at other suitable substrates (e.g., slides, plates, etc.) functionalized with material components structured to interact with target material of the sample. For instance, sample material can be spotted onto substrates with material components structured to interact with target material of the sample and in a detectable manner.

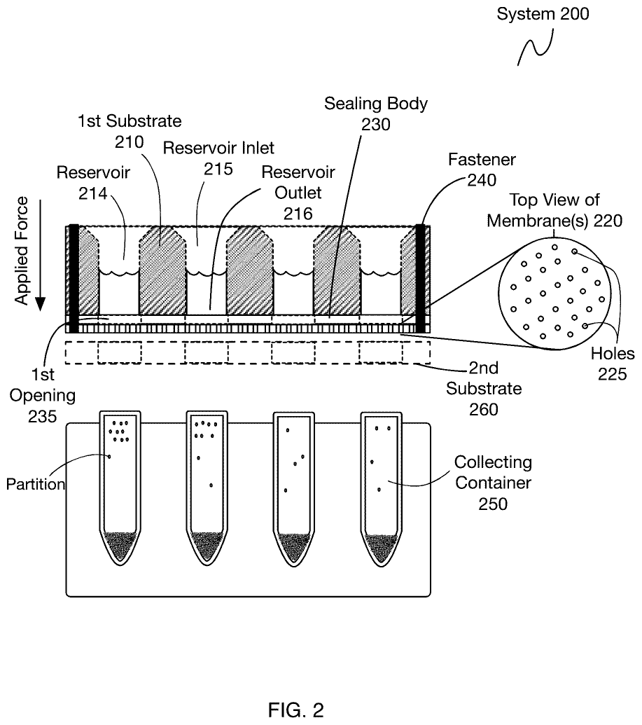

Embodiments, variations, and examples of the methods described can be implemented by or by way of embodiments, variations, and examples of components of system 200 shown in FIG. 2, with a first substrate 210 defining a set of reservoirs 214 (for carrying sample/mixtures for droplet generation), each having a reservoir inlet 215 and a reservoir outlet 216; one or more membranes (or alternatively, droplet-generating substrates) 220 positioned adjacent to reservoir outlets of the set of reservoirs 214, each of the one or more membranes 220 including a distribution of holes 225; and optionally, a sealing body 230 positioned adjacent to the one or more membranes 120 and including a set of openings 235 aligned with the set of reservoirs 214; and optionally, one or more fasteners (including fastener 240) configured to retain the first substrate 210, the one or more membranes 220, and optional sealing body 230 in position relative to a set of collecting containers 250. In variations, the system 200 can additionally include a second substrate 260, wherein the one or more membranes 220 and optionally, the sealing body 230, are retained in position between the first substrate 210 and the second substrate 260 by the one or more fasteners. While using embodiments, variations, and examples of the system 200, material derived from each sample is retained in its own tube and does not require batching and pooling, allowing for scalable batch sizes.

In variations, the distribution of holes 225 can be generated in bulk material with specified hole diameter(s), hole depth(s) (e.g., in relation to membrane thickness), aspect ratio(s), hole density, and hole orientation, where, in combination with fluid parameters, the structure of the membrane can achieve desired flow rate characteristics, with reduced or eliminated polydispersity and merging, suitable stresses (e.g., shear stresses) that do not compromise nucleic acid material, single cells, or other materials, but allow for partitioning, and steady formation of droplets (e.g., without jetting of fluid from holes of the membrane).

In variations, the hole diameter can range from 0.01 micrometers to 30 micrometers, and in examples, the holes can have an average hole diameter of 0.01 micrometers, 0.02 micrometers, 0.04 micrometers, 0.06 micrometers, 0.08 micrometers, 0.1 micrometers, 0.5 micrometers, 1 micrometers, 2 micrometers, 3 micrometers, 4 micrometers, 5 micrometers, 6 micrometers, 7 micrometers, 8 micrometers, 9 micrometers, 10 micrometers, 20 micrometers, 30 micrometers, any intermediate value, or greater than 30 micrometers (e.g., with use of membrane having a thickness greater than or otherwise contributing to a hole depth greater than 100 micrometers).

In variations, the hole depth can range from 1 micrometer to 200 micrometers (e.g., in relation to thickness of the membrane layer) or greater, and in examples the hole depth (e.g., as governed by membrane thickness) can be 1 micrometers, 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or any intermediate value.

In variations, the hole aspect ratio can range from 5:1 to 200:1, and in examples, the hole aspect ratio can be 5:1, 10:1, 20:1, 30:1, 40:1, 50:1, 60:1, 70:1, 80:1, 90:1, 100:1, 125:1, 150:1, 175:1, 200:1, or any intermediate value.

In variations, the hole-to-hole spacing can range from 5 micrometers to 200 micrometers or greater, and in examples, the hole-to-hole spacing is 5 micrometers, 10 micrometers, 20 micrometers, 30 micrometers, 40 micrometers, 50 micrometers, 60 micrometers, 70 micrometers, 80 micrometers, 90 micrometers, 100 micrometers, 125 micrometers, 150 micrometers, 175 micrometers, 200 micrometers, or greater. In a specific example, the hole-to-hole spacing is greater than 10 micrometers.

In examples, the hole orientation can be substantially vertical (e.g., during use in relation to a predominant gravitational force), otherwise aligned with a direction of applied force through the distribution of holes, or at another suitable angle relative to a reference plane of the membrane or other droplet generating substrate 220.

In examples, the system 200 can process an input sample, with high performance in relation to dead volume (e.g., volume of sample that is not dropletized for further processing). In examples, the dead volume of a sample processed by the system 200 is less than 8%, less than 7%, less than 6%, less than 5%, less than 4%, less than 3%, less than 2%, or a lower percentage of the volume of the input sample.

Additionally or alternatively, embodiments, variations, and examples of the methods described can be implemented by or by way of embodiments, variations, and examples of components described in U.S. application Ser. No. 17/687,080 filed 4 Mar. 2022, U.S. Pat. No. 11,242,558 granted 8 Feb. 2022, U.S. application Ser. No. 16/309,093 filed 25 May 2017, and PCT Application PCT/CN2019/093241 filed 27 Jun. 2019, each of which is herein incorporated in its entirety by this reference. As such, generating droplets can include transmitting sample material through one or more fluid layers (e.g., including air, aqueous fluids, non-aqueous fluids, etc.), to generate an emulsion having suitable clarity (e.g., without refractive index matching). However, methods described can additionally or alternatively implement other system elements for sample reception and processing.

### 2.3 Method-Amplification

Step S120 recites: amplifying the set of nucleic acid molecules within individual partitions of the set of partitions for a set of amplification cycles, such that the partition in the representative state contains a set of amplicons generated from the single nucleic acid molecule. Amplification in operation S120 is performed using a heating subsystem in communication with a controller of a computing subsystem, where computing and processing architecture are described further in Section 4 below. In variations, operation S120 can include transporting the collecting container(s) (e.g., containers of the set of partitions, containers of droplets of emulsions generated from input sample material) or receiving the collecting containers at the heating subsystem, and then initiating an amplification protocol for transmitting heat to and/or from the collecting containers. In variations, transmitting the collecting containers can include automatically transmitting the collecting containers with robotic apparatus in communication with a controller. In variations, transmitting the collecting containers can include manually transmitting the collecting containers (e.g., by an operator or other entity).

Amplification according to operation S120 can involve PCR-based methods or non-PCR based methods. Amplification of partitioned nucleic acid molecules can include exponential amplification of partitioned nucleic acid molecules. Amplification of the labeled nucleic acids can include linear amplification or otherwise non-exponential amplification of partitioned nucleic acid molecules.

In variations, amplification of partitioned nucleic acid molecules can include non-PCR based methods. Example non-PCR based methods can include amplification methods derived from: multiple displacement amplification (MDA), other isothermal amplification methods, transcription-mediated amplification (TMA), nucleic acid sequence-based amplification (NASBA), strand displacement amplification (SDA), real-time SDA, rolling circle amplification, circle-to-circle amplification, and other non-PCR-based amplification methods (e.g., multiple cycles of DNA-dependent RNA polymerase-driven RNA transcription amplification, RNA-directed DNA synthesis and transcription to amplify DNA or RNA targets, etc.).

As such, in relation to use of non-isothermal and isothermal methods of amplification, the set of amplification cycles (e.g., of a PCR protocol) can include one amplification cycle or greater than one amplification cycle (e.g., 2 amplification cycles, 3 amplification cycles, 4 amplification cycles, 5 amplification cycles, 6 amplification cycles, 7 amplification cycles, 8 amplification cycles, 9 amplification cycles, 10 or more amplification cycles, 20 or more amplification cycles, 30 or more amplification cycles, 40 or more amplification cycles, 50 or more amplification cycles, etc.). Amplification in operation S120 can further involve one or more pre-amplification cycles.

Amplification protocols in operation S120 can implement temperature ramp-up rates, temperature ramp-down rates, temperature cycling profiles, and/or temperature holding profiles. Temperatures for amplification can range between 25° C. and 99° C. (or alternative temperatures). Holding temperatures can range between 25° C. and 99° C. (or alternative temperatures). Storage temperatures can range between 0° C. and 20° C. (or alternative temperatures).

Each amplification cycle can range between 10 seconds and 15 minutes or greater (or alternative time durations) for non-isothermal amplification methods. As such, an amplification cycle duration can be greater than 10 seconds, greater than 20 seconds, greater than 30 seconds, greater than 40 seconds, greater than 50 seconds, greater than 1 minute, greater than 2 minutes, greater than 3 minutes, greater than 4 minutes, greater than 5 minutes, greater than 6 minutes, greater than 7 minutes, greater than 8 minutes, greater than 9 minutes, greater than 10 minutes, greater than 15 minutes, or greater.

For isothermal amplification methods/protocols, an amplification cycle can range from 30 minutes to 20 hours or greater (or alternative time durations). As such, the amplification cycle duration can be greater than 30 minutes, greater than 1 hour, greater than 2 hours, greater than 3 hours, greater than 4 hours, greater than 5 hours, greater than 6 hours, greater than 7 hours, greater than 8 hours, greater than 9 hours, greater than 10 hours, greater than 15 hours, greater than 20 hours, or greater.

Example amplification protocols for specific cases of use (e.g., library preparation for immune repertoire sequencing, library preparation for single cell whole genome characterizations, library preparation for pathogen panel sequencing, library preparation for microbiome analyses, etc.) are described further in Section 3 below.

### 2.4 Method-Purification

Step S130 recites: performing a nucleic acid purification operation upon amplicons of partitions of the set of partitions. Step S130 functions to execute nucleic acid cleanup operations that isolate and purify nucleic acid material after amplification, with removal of non-desired sample materials. Processes performed prior to the nucleic acid purification operation can include operations for deactivating polymerases used in amplification (e.g., with a temperature hold to deactivate polymerases), and retrieving amplified nucleic acids from partitions.

In variations where the partitions are droplets of an emulsion, retrieving amplified nucleic acids can include disrupting the emulsion to release the amplified and tagged nucleic acid material, and removal of non-aqueous components of the emulsion. Disrupting emulsions can be performed by mixing the emulsion with an emulsion breaking reagent (e.g., 1-Butanol, a butanol reagent, water, a substance that disrupts an equilibrium state of the emulsion, etc.), for instance, with multiple aspirations and deliveries of the emulsion with the emulsion breaking reagent (e.g., using a manual pipettor, using a pipetting head, etc.) or agitation/rocking/vibration of a mixture of the emulsion with the emulsion breaking reagent. As such, the method can include disrupting the emulsion with a butanol reagent, thereby releasing said amplicons from the set of droplets prior to performing the nucleic acid purification operation.

Removal of the non-aqueous components of the emulsion can be performed post-centrifugation of the mixture of the emulsion with the emulsion breaking reagent, to facilitate non-aqueous component removal. Alternatively, disrupting emulsions can be performed without use of an emulsion-breaking reagent, and involve only mechanical disruption (e.g., by agitation, by mixing, by rocking, by vibration, etc.).

Purification in operation S130 is performed using fluid handling apparatus (e.g., of a fluid handling subsystem) and separation apparatus (e.g., of a magnetic separation subsystem, of a purification subsystem, of a buoyant particle separation subsystem, etc.) in communication with a controller of a computing subsystem, where computing and processing architecture are described further in Section 4 below. In variations of operation S120, the fluid handling apparatus can transport one or more volumes of fluids containing amplicons produced in operation S110 from the collecting container(s) to the separation apparatus for performance of the nucleic acid purification operation. Transport can be performed manually (e.g., by an operator or other entity) or non-manually (e.g., using robotic apparatus for transport of volumes).

In variations, the separation apparatus can operate by way of one or more of: magnetic separation (e.g., using magnetic particles in a solid phase reversible immobilization (SPRI) technique, using other functionalized magnetic particles), column-based separation (e.g., chromatographic methods using membrane columns and a chaotropic salt, other column-based methods of separation), buoyancy-based separation (e.g., with buoyant particles that capture nucleic acids and are separatable from fluids based on buoyancy). Separation in operation S130 can involve materials that bind and remove target nucleic acid molecules or fragments from other sample materials. Separation in operation S130 can involve materials that bind and remove non-target components of a sample.

The nucleic acid purification operation of operation S130 can include one or more mixing and/or washing operations, in order to purify amplified nucleic acid molecules in one or more stages.

Example purification protocols for specific cases of use (e.g., library preparation for immune repertoire sequencing, library preparation for single cell whole genome characterizations, library preparation for pathogen panel sequencing, library preparation for microbiome analyses, etc.) are described further in Section 3 below.

### 2.5 Method—Library Preparation

Step S140 recites: returning a prepared nucleic acid library upon performance of the nucleic acid purification operation, where the prepared nucleic acid library comprises an amplification bias below a first threshold level and a percentage of chimeric sequences below a second threshold level. Step S140 functions to provide a collection of nucleic acid molecules or fragments that are purified to a desired state, and transferable for further analysis and processing (e.g., by sequencing). The prepared nucleic acid library can be stabilized and stored in a desired environment.

In examples, stabilization and storage can involve a desired buffer, with storage at 4° C. or at another suitable temperature, until further processing.

In variations of operation S140, the first threshold level can be a threshold level of 10-fold reduction in amplification bias, 50-fold reduction in amplification bias, 100-fold reduction in amplification bias, 500-fold reduction in amplification bias, 1000-fold reduction in amplification bias, 5000-fold reduction in amplification bias, 10000-fold reduction in amplification bias, or greater reduction in amplification bias, in comparison to current technologies for library preparation.

Determining the level of amplification bias can involve: addition of unique molecule identifiers (UMIs) to primers used for amplification, followed by comparing the variation of reads per UMI used upon reading sequences of amplicons generated according to methods described, in comparison to sequences of amplicons generated using another method (e.g., bulk amplification without partitioning, bulk amplification without partitioning according to methods described, etc.). In one example, determining the level of amplification bias can include: performing one or more cycles of PCR to tag partitioned nucleic acids with 10-base UMIs (using forward and reverse primers), amplifying UMI-tagged nucleic acids, and performing nucleic acid purification operation with nucleic acid titration to obtain sufficient quantities of nucleic acids for sequencing in relation to library preparation.

In particular, determining the level of amplification bias can involve determining first relative distribution values of a subset of input molecules of the set of nucleic acid molecules prior to amplification, and determining second relative distribution values of amplicons of the subset of input molecules after amplification, such that the first threshold level against which the level of amplification bias is compared represents: a less than 20% difference between said first relative distribution values and said second distribution abundance values, a less than 19% difference between said first relative distribution values and said second relative distribution values, a less than 18% difference between said first relative distribution values and said second relative distribution values, a less than 17% difference between said first relative distribution values and said second relative distribution values, a less than 16% difference between said first relative distribution values and said second relative distribution values, a less than 15% difference between said first relative distribution values and said second relative distribution values, a less than 14% difference between said first relative distribution values and said second relative distribution values, a less than 13% difference between said first relative distribution values and said second relative distribution values, a less than 12% difference between said first relative distribution values and said second relative distribution values, a less than 10% difference between said first relative distribution values and said second relative distribution values, a less than 9% difference between said first relative distribution values and said second relative distribution values, a less than 8% difference between said first relative distribution values and said second relative distribution values, a less than 7% difference between said first relative distribution values and said second relative distribution values, a less than 6% difference between said first relative distribution values and said second relative distribution values, a less than 5% difference between said first relative distribution values and said second relative distribution values, a less than 4% difference between said first relative distribution values and said second relative distribution values, a less than 3% difference between said first relative distribution values and said second relative distribution values, a less than 2% difference between said first relative distribution values and said second relative distribution values, or lower.

The UMIs can be added to forward and/or reverse primers used for amplification, and can include 5 bases, 6 bases, 7 bases, 8 bases, 9 bases, 10 bases, 11 bases, 12 bases, 13 bases, 14 bases, 15 bases, or greater numbers of bases.

In variations of operation S140, the second threshold level can be a threshold level of: less than 15% representation by artifact sequences in the prepared nucleic acid library, less than 14% representation by artifact sequences in the prepared nucleic acid library, less than 13% representation by artifact sequences in the prepared nucleic acid library, less than 12% representation by artifact sequences in the prepared nucleic acid library, less than 11% representation by artifact sequences in the prepared nucleic acid library, less than 10% representation by artifact sequences in the prepared nucleic acid library, less than 9% representation by artifact sequences in the prepared nucleic acid library, less than 8% representation by artifact sequences in the prepared nucleic acid library, less than 7% representation by artifact sequences in the prepared nucleic acid library, less than 6% representation by artifact sequences in the prepared nucleic acid library, less than 5% representation by artifact sequences in the prepared nucleic acid library, less than 4% representation by artifact sequences in the prepared nucleic acid library, less than 3% representation by artifact sequences in the prepared nucleic acid library, less than 2% representation by artifact sequences in the prepared nucleic acid library, less than 1% representation by artifact sequences in the prepared nucleic acid library, or lower levels of sequences in a generated library.

Prepared library outputs of operation S140 can also be be characterized by false positive sequences reduced by a factor of 10-fold, 50-fold, 100-fold, 500-fold, 1000-fold, 5000-fold, 10000-fold, or greater, in comparison to current technologies for library preparation involving bulk amplification.

Example aspects of library preparation for immune repertoire sequencing, library preparation for single cell whole genome characterizations, library preparation for pathogen panel sequencing, and library preparation for microbiome analyses, are described further in Section 3 below.

### 2.6 Method-Adapters for Sequencing Platforms

In variations, the method 100 can further include adding a set of adapter sequences in coordination with amplification, the set of adapter sequences corresponding to a sequencing platform. Step S150 functions to ligate adapters to amplicons generated according to methods described, in order to facilitate flow cell sequencing-based operations or otherwise ensure sequencing platform-specific compatibility.

Adapters can include adapters for NGS platforms, and example adapters can include TruSeq™ adapters (e.g., full-length adapters and/or indexing primers, stubby adapters and/or indexing primers), Nextera™ adapters and/or indexing primers, adapters for methylated bases, Illumina™ adapter sequences (e.g., i5 sequences, i7 sequences, etc.) for use with iSeq 100™, NovaSeq 6000™, MiniSeq™, NextSeq 2000™, NextSeq 550™, NextSeq 500™, HiSeq 4000™, HiSeq 3000™, HiSeq X™, or other adapters.

Adapters can include barcode/index sequences to facilitate identification of samples and enable multiplexing in target enrichment and sequencing. Indices can include combinatorial indices and/or dual indices. Adapters and Indices used can be designed for various indexing strategies, for instance, for broad-range RNA library preparation, RNA library preparation, DNA library preparation, low-input DNA library preparation, ssDNA library preparation, methyl-seq library preparation, cfDNA library preparation, formalin-fixed and parafin-embedded (FFPE) DNA library preparation, and/or other strategies.

### 3. Specific Applications

### 3.1 Specific Application-Immune Repertoire Sequencing

In a specific use case, systems, methods, and compositions described were used for immune repertoire characterization (e.g., in relation to VDJ recombination detection of leading peptide, variable, diversity, joining, and/or constant genomic segments, in relation to TCR repertoires, in relation to BCR repertoires, etc.), where current sequencing technologies are fraught with high false positive rates and/or high PCR error. In relation to the specific use case, systems, methods, and compositions described can be used to disperse a sample of T-cell receptor (TCR), B-cell receptor (BCR), and/or other genetic material across a plurality of partitions (as described above, with example occupancy of partitions by targets at a rate of 40-100%), where processing materials described enable detection of TCR and/or BCR characteristics, including VDJ recombination/rearrangement assessment (e.g., quantification of VDJ targets), for immune system characterization, disease tracking, monitoring responses to therapy, and/or other aspects associated with the sample. As such, the sample can include T-cell receptor material, and the set of nucleic acid molecules can include variable, diversity, and joining (VDJ) sequences for library preparation. Examples of methods described can accurately return nucleic acid libraries, followed by sequencing to return an analysis of a set of TCR and/or BCR characteristics, including a VDJ recombination/rearrangement assessment with parameter values indicating VDJ recombinations and rearrangements.

An example method for library preparation for immune repertoire characterization involved: generating a dilution of DNA molecules (e.g., a 1:1.19 dilution of peripheral blood mononuclear cell cDNA molecules, a dilution of human donor T/B cell RNA with cell lines such as Jurkat, Ramos, etc.); combining the dilution with a master mixture (e.g., a master mixture including PCR certified water, dPCR buffer, dNTPs, primers, and Taqman™ Epimark polymerase) as one or more input samples; partitioning the input sample(s) with partitioning apparatus as described above to generate one or more emulsions; amplifying partitioned nucleic acid molecules with a thermocycling operation (e.g., a thermocycling operation including an initial denaturation cycle at 95° C. for 2 minutes; 10 additional cycles with a denaturation cycle at 95° C. for 30 seconds for each cycle, an annealing cycle at 60° C. for 30 seconds each cycle, and an extension cycle at 68° C. for 60 seconds each cycle; a final extension cycle at 65° C. for 5 minutes; and storage at 12° C.); disrupting emulsions with butanol (e.g., breaking emulsions with amplicons, by mixing the emulsions with butanol repeatedly, centrifuging broken emulsions, and removing non-aqueous emulsion components to generate a sample for purification); performing a nucleic acid purification operation by combining magnetic SPRI beads with the sample for purification and capturing nucleic acids with the magnetic SPRI beads (e.g., upon centrifuging the SPRI bead sample and removing a supernatant); washing retrieved SPRI beads with captured nucleic acid content (e.g., with ethanol), and repeating separation and washing one or more times; and returning a prepared nucleic acid library.

Variations of the method for library preparation for immune repertoire characterization can accept different amounts of input RNA and/or involve other amplification protocols (e.g., a thermocycling operation including an initial denaturation cycle at 98° C. for 2 minutes; additional cycles with a denaturation cycle at 98° C. for 20 seconds for each cycle, an annealing cycle at 65° C. for 45 seconds each cycle, and an extension cycle at 72° C. for 3 minutes each cycle; a final extension cycle at 72° C. for 5 minutes; and storage at 4° C.), with targeted amplification of the VDJ region of immune receptors.

Representative results of methods described significantly reduce or eliminate PCR bias and chimera/artifact generation during sample processing, to achieve greater than 70% accuracy, greater than 80% accuracy, greater than 90% accuracy, greater than 91% accuracy, greater than 92% accuracy, greater than 93% accuracy, greater than 94% accuracy, greater than 95% accuracy, greater than 96% accuracy, greater than 97% accuracy, greater than 98% accuracy, or greater than 99% accuracy in VDJ-seq results. Furthermore, in relation to immune repertoire sequencing of the VDJ junction (VDJ-seq), the disclosure provides methods, systems, and compositions that are not subject to overestimations of receptor frequencies, where standard technologies inherently overestimate receptor frequency by up to 5000-fold.

Representative results of methods described significantly reduce eliminate chimera/artifact generation during sample processing, such that chimeric/artifact sequences represent: less than 15% representation by artifact sequences in the prepared nucleic acid library, less than 14% representation by artifact sequences in the prepared nucleic acid library, less than 13% representation by artifact sequences in the prepared nucleic acid library, less than 12% representation by artifact sequences in the prepared nucleic acid library, less than 11% representation by artifact sequences in the prepared nucleic acid library, less than 10% representation by artifact sequences in the prepared nucleic acid library, less than 9% representation by artifact sequences in the prepared nucleic acid library, less than 8% representation by artifact sequences in the prepared nucleic acid library, less than 7% representation by artifact sequences in the prepared nucleic acid library, less than 6% representation by artifact sequences in the prepared nucleic acid library, less than 5% representation by artifact sequences in the prepared nucleic acid library, less than 4% representation by artifact sequences in the prepared nucleic acid library, less than 3% representation by artifact sequences in the prepared nucleic acid library, less than 2% representation by artifact sequences in the prepared nucleic acid library, less than 1% representation by artifact sequences in the prepared nucleic acid library, or lower levels of sequences in a generated library.

### 3.2 Specific Application-Single Cell Whole Genome Sequencing

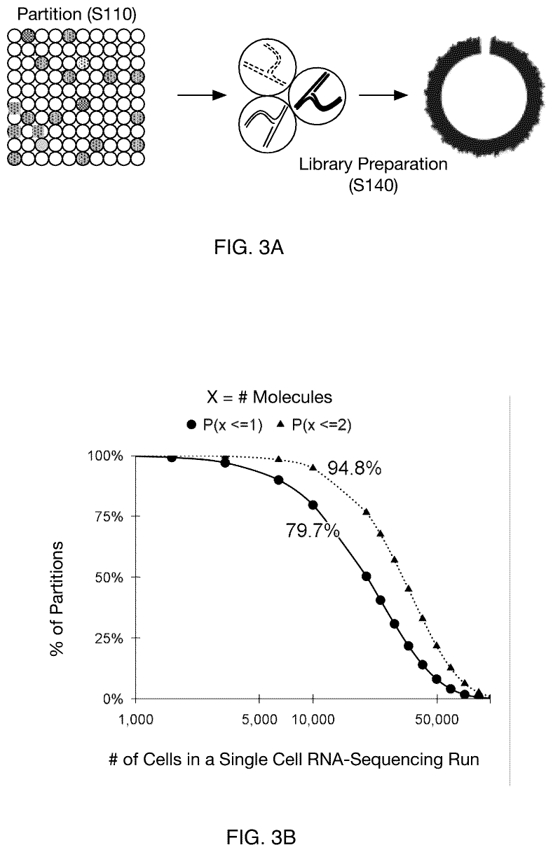

In another specific use case (shown in FIGS. 3A and 3B), systems, methods, and compositions described were used for single cell whole genome characterization, where current sequencing technologies are fraught with high PCR bias. In relation to the specific use case, systems, methods, and compositions described were used to disperse a sample of single cells and/or single cell genetic material (e.g., single cell genome fragments) across a plurality of partitions (as described, with expected 40-70% occupancy), where processing materials described function to barcode single cell material within partitions (e.g., with stochastic barcodes, with other barcodes, etc.), and enable detection of single cell genome components. The specific example involved multiple displacement amplification (MDA), which typically harbors high amplification bias; however, representative results demonstrated significantly reduced levels of amplification bias in comparison to bulk amplification methods.

In variations, a sample can include materials from single cells, and the set of nucleic acid molecules comprises single cell whole genome sequences for library preparation.

An example method for library preparation for single cell whole genome sequencing involved: generating a sample of nucleic acids from T-cells (e.g., human T-cells) upon lysing the cells and denaturing DNA from the lysed cells; combining the sample with a master mixture (e.g., a master mixture including phi29 buffer, dNTPs, heaxamer, phi29 polymerase, and DMSO, with a Repli-g™ MDA kit) as one or more input samples, where the phi29 polymerase can be added to the master mixture and to the denatured DNA in order to prevent premature polymerase activity; partitioning the input sample(s) with partitioning apparatus as described above to generate one or more emulsions; amplifying partitioned nucleic acid molecules (e.g., with an amplification operation including isothermal amplification at 30° C. for 12 hours (at least 10 hours); a polymerase deactivation operation at 65° C. for 10 minutes; and storage at 4° C.); disrupting emulsions with butanol (e.g., breaking emulsions with amplicons, by mixing the emulsions with butanol repeatedly, centrifuging broken emulsions, and removing non-aqueous emulsion components to generate a sample for purification); performing a nucleic acid purification operation by combining magnetic SPRI beads with the sample for purification and capturing nucleic acids with the magnetic SPRI beads (e.g., upon centrifuging the SPRI bead sample and removing a supernatant); washing retrieved SPRI beads with captured nucleic acid content (e.g., with ethanol), and repeating separation and washing one or more times; and returning a prepared nucleic acid library.

Furthermore, in addition to using sequencing as a read out, methods described can additionally include performance of amplification (e.g., Taqman™ PCR), performance of digital PCR, use of microarrays, use of bead arrays, and/or other methods of assessing library representation.

Representative results of methods described significantly reduce or eliminate PCR bias and chimera/artifact generation during sample processing, to achieve greater than 70% accuracy, greater than 80% accuracy, greater than 90% accuracy, greater than 91% accuracy, greater than 92% accuracy, greater than 93% accuracy, greater than 94% accuracy, greater than 95% accuracy, greater than 96% accuracy, greater than 97% accuracy, greater than 98% accuracy, or greater than 99% accuracy in single cell whole genome library preparation results.

Variations of single cell whole genome amplification described, can additionally or alternatively have applications in vitro fertilization, where partitioned, processed, and amplified input materials can be used to assessing embryo quality, sperm quality, and other factors affecting outcomes in in vitro fertilization.

3.2.1 Specific Application-Single Cell RNA-Seq and Library Preparation with Barcoding

Variations of the methods can be applied to single cell RNA sequencing (single cell RNA-seq) and/or related library preparation methods from single cell sample material, using a using a barcoding scheme, In more detail, cellular content of the input sample can be barcoded, and tagged with certain specific universal sequences, followed by universal amplification using a set of primers. In this regard, emulsion amplification (as described in relation to sample distribution and amplification methods described above) can reduce amplification bias and chimera formation, and allow each molecule to be amplified equally, such that a lower amount of sequencing can be performed to recover a majority of the molecules and increase the recovery rate of molecules of the input sample.

As such, the method(s) can additionally or alternatively able to accurately generate and amplify single cell RNA-seq libraries, in order to preserve the original representation of the library (e.g., the original representation of distributions present in the original input sample) and reducing the amount of sequencing required in order to discover all molecules present. In particular, some molecules of an input sample may be amplified more efficiently than others, so compared to methods as described herein, other methods may require more significant sequencing efforts in order to discover those molecules that are amplified less relative to other molecules of the input sample.

Processing materials that are combined with single cell materials for RNA-seq and/or related library preparation methods can include components for barcoding/tagging of single cells. For instance, processing materials can include cellular and/or molecular labels (e.g., oligonucleotide sequences), where a cellular label can include a nucleic acid sequence (e.g., a random nucleic acid sequence) that provides information regarding a single cell that interacted with the cellular label. A cellular label can have a length of 1, 2, 3, 4, 5, 10, 15, 20, 25, 30, 35, 40, 45, 50 or more nucleotides. A cellular label can have a length of at most about 300, 200, 100, 90, 80, 70, 60, 50, 40, 30, 20, 15, 12, 10, 9, 8, 7, 6, 5, 4 or fewer or more nucleotides.

A molecular label can include a nucleic acid sequence (e.g., a random nucleic acid sequence) that informs the specific nucleic acid species hybridized to the oligonucleotide. In this way, the molecular label may distinguish different target nucleic acids that are present within a droplet/partition. A molecular label can have a length of at least 1, 2, 3, 4, 5, 10, 15, 20, 25, 30, 35, 40, 45, 50 or more nucleotides. A molecular label can have a length of: at most 300, 200, 100, 90, 80, 70, 60, 50, 40, 30, 20, 15, 12, 10, 9, 8, 7, 6, 5, 4 or fewer nucleotides.

Oligonucleotides can further include sample indexing regions, capture regions (e.g., mRNA capture regions with poly A/polyT capture functionality), linker regions (e.g., cleavable linker regions), adapter regions, extension regions, primer binding sequences, and/or other functional regions.

Labelling can be performed with use of functionalized particles that are combined with the sample and/or distributed across partitions, where such partitions contain material from single cells individually, and where each functionalized particle can have the same cellular label, but different molecular labels. Functionalized particles can be magnetic or non-magnetic. Functionalized particles can be porous or non-porous. Functionalized particles can be buoyant or have suitable density properties. Functionalized particles can be configured to swell (e.g., as in a hydrogel within an aqueous solution), or can be configured to not swell in certain environments. Functionalized particles can be configured to controllably degrade in certain environmental conditions.

### 3.3 Specific Application—16S and 18S Library Preparation

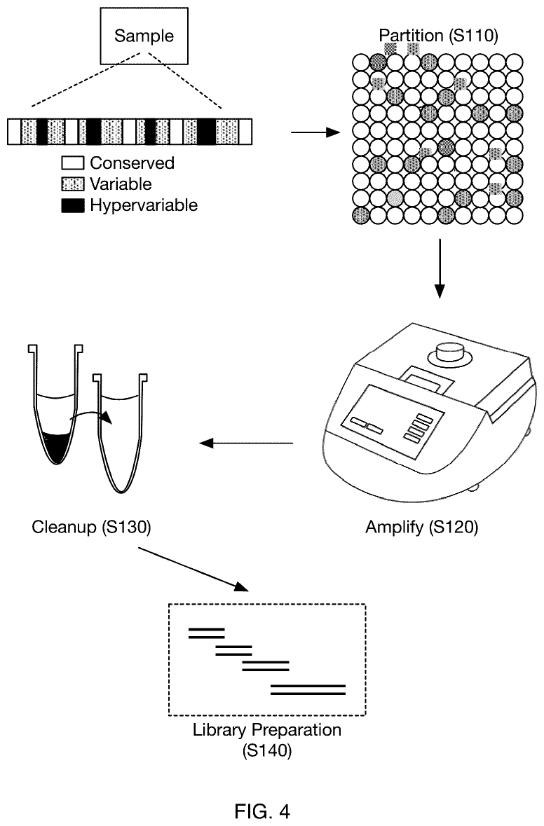

In another specific use case (shown in FIG. 4), systems, methods, and compositions described can be useful for ribosomal 16S, 18S, and/or ITS characterization, where current sequencing technologies are fraught with high false positive rates and/or high PCR error. In relation to the specific use case, systems, methods, and compositions described can be used to disperse a sample of 16S and/or 18S and/or ITS ribosomal RNA (rRNA) across a plurality of partitions (as described), where processing materials described enable library preparation for regions/sequences of interest (e.g., V3 region, V4 region, V5, region, other hypervariable regions, etc.), and subsequently, for operational taxonomic unit (OTU) or amplicon sequence variant (ASV) categorizations. For instance, detection of V3, V4, and/or V5 regions can be used for bacterial microbiome analyses, fungal microbiome analyses, other microbiome analyses, rare species detection, and/or other applications. Additionally or alternatively, such rRNA characterizations can be used for detection of a set of pathogens (e.g., up to 30 pathogens, up to 40 pathogens, up to 50 pathogens, up to 60 pathogens, up to 70 pathogens, etc.). Additionally or alternatively, for microbial pathogen detection/quantification, any part of microbial genomics of a sample (e.g., non-rRNA targets) can be targeted. In variations, the sample can include ribosomal RNA (rRNA) of a microbial material sample, and the set of nucleic acid molecules comprises at least one of 16S and ITS rRNA sequences for library preparation.

An example workflow implements multiple ribosomal 16S and 18S sequencing methods (e.g., with a Quick-16S NGS Library Prep Kit (Zymo Research™), with Illumina™ Library Preparation materials), with comparison of species representation and chimera formation from bulk amplification workflows in comparison to workflows using the inventions described. The example method involved a control mixture of known bacterial species (e.g., ATCC 14990, ATCC 4505, and ATCC 10700). After genomic DNA extraction, the example workflow involved quantifying the amount of material for each bacterial sample, prior to mixing them in a defined ratio (e.g., 1:1:1, other ratios) in order to enable direct comparisons of results from different workflows. The example method then included performance of targeted sequence amplification, enzymatic clean up operations, barcode addition, library quantification and pooling, and a consolidated library clean up operation.

Representative results of methods described reduce chimera and other artifact production during library preparation, such that artifacts/chimeras represent less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library, thereby improving accuracy of analyses of diversity (e.g., microbiome diversity) within a sample. An example use case involves analyses of samples involving hypervariable regions of rRNA (e.g., v3-v5 regions of 16S rRNA). In particular, accurate library preparation for microbiome samples results in improved accuracy of diversity analyses, abundance inferences, and other important metrics for characterizations.

### 3.4 Specific Application-Liquid Biopsy and Precision Diagnostics

In relation to applications in precision diagnostics, the disclosure provides methods, systems, and compositions that are capable of accurate library preparation for samples involving extremely small allelic fractions, such as allelic fractions less than 1%, less than 0.9%, less than 0.8%, less than 0.7%, less than 0.6%, less than 0.4%, less than 0.3%, less than 0.2%, less than 0.1%, less than 0.05%, or less.

Applications of use include library preparation for detection of oncogenic gene fusions (e.g., anaplastic lymphoma kinase (ALK) fusions, BCR-ABL fusions, etc.). Accuracy is enhanced due to aspects of the invention(s) that result in artifacts/chimeras representing less than 15%, less than 14%, less than 13%, less than 12%, less than 11%, less than 10%, less than 9%, less than 8%, less than 7%, less than 6%, less than 5%, less than 4%, less than 3%, less than 2%, less than 1%, or lower levels of sequences in a generated library, as well as reductions in false-positive rates by a factor of 10-fold, 50-fold, 100-fold, 500-fold, 1000-fold, 5000-fold, 10000-fold, or greater. In particular, accurate library preparation for samples results in improved accuracy of, abundance inferences associated with copy number variations, and other important metrics for characterizations.

In relation to liquid biopsy, an example workflow involved cfDNA library preparation, with different target panel sizes. Example methods implemented mixtures of nucleosomal DNA from various cell lines, (e.g., 1% of a cell line with known oncogenic mutations targeted by the panels into a background normal cell line (e.g., GM12878), in order to mimic cell-free DNA from cancer patients).

For a first kit, the example workflow involved processing reagents with 20 ng of DNA template and distributing combined reagents and samples across partitions (e.g., using methods described), followed by an amplification operation performed according to kit recommendations.

For a second kit, the example workflow involved a larger panel size (81 kb) for an alternative LP method which uses biotinylated bait capture enrichment method that requires 2 PCR amplification operations—a pre-enrichment PCR operation and post-enrichment PCR operation. The pre-enrichment PCR operation performs whole genome amplification, whereby all adapter-ligated cfDNA fragments have an equal chance to be amplified. The pre-enrichment PCR produced approximately 7.13×10¹⁰ templates per 25 ng cfDNA with 100% adapter ligation efficiency, which rendered an average of 200 DNA template copies per droplet. The post-enrichment PCR operation provided a capture efficiency of 50%, producing 9.6×10⁵ template copies, which rendered an average of 0.032 DNA copies per droplet.

The example workflows thus characterize potential benefits of performing library preparation as described, including, but not limited to: Library uniformity attributes (e.g., as measured by the uniformity of read coverage of reads with different % GC content, as well as reads per molecular barcode uniformity); Representation of rare transcripts by examining whether rare mutations spiked into the DNA mixture are better represented; Chimera formation by examining reads that map to >1 location and is not a characterized translocation event in the cell lines; and other benefits.

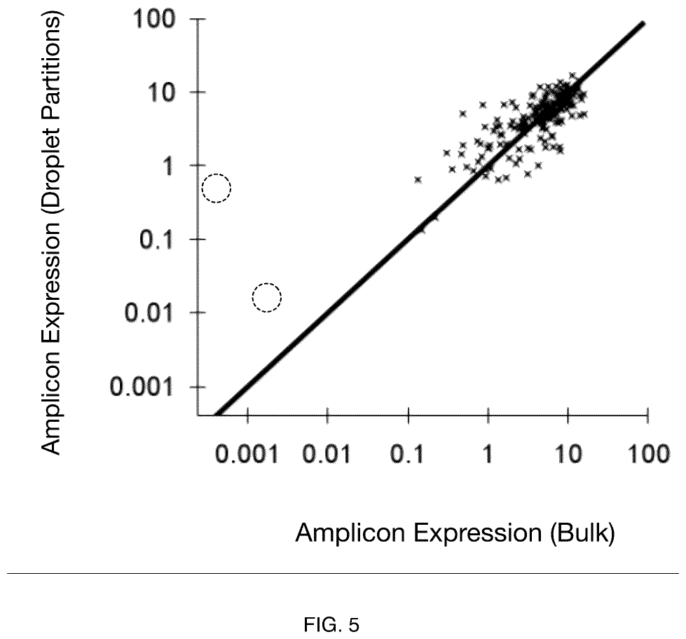

Relatedly, example FIG. 5 depicts outputs of library preparation processes using a dropletized partition workflow in comparison to a ‘bulk’ amplification workflow targeting 180 loci, using a 25 ng nucleosomal DNA input purified from a GM12878 cell line using identical reagents. The libraries were then sequenced on a next generation sequencing platform. Amplicon counts in FIG. 5 are normalized to the 1000 total reads mapped to amplicon reference file before comparison. The solid black line represents y=x to indicate the expected amplicon counts based on the bulk amplification workflow. The dashed circles represents two loci with low expression in bulk amplification samples and higher expression with use of the partitioning-based approach described (thereby demonstrating significantly reduced amplification bias). R2=0.461.

### 4. Computer Systems

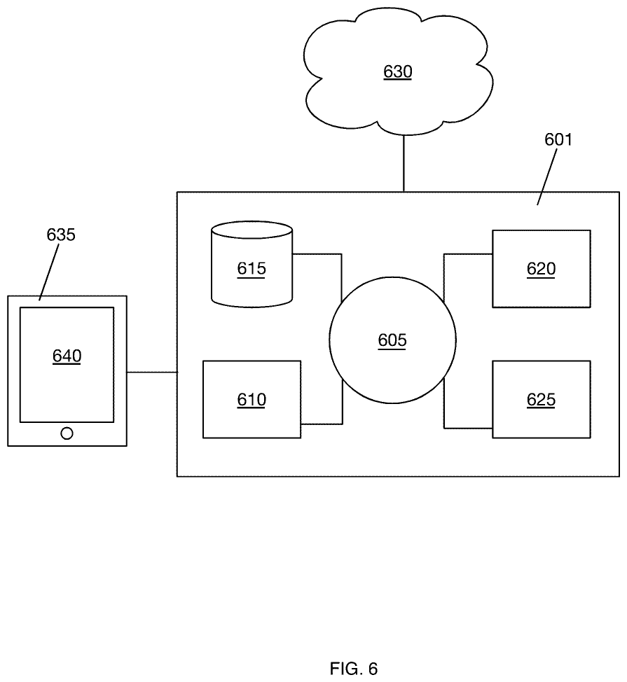

The present disclosure provides computer systems that are programmed to implement methods of the disclosure (e.g., in coordination with or by providing instructions to one or more controllers of various apparatus). FIG. 6 shows a computer system 601 that is programmed or otherwise configured to, for example, generate nucleic acid libraries in a highly accurate manner, by enabling one or more of: distributing a sample comprising a set of nucleic acid molecules across a set of partitions at low occupancy (e.g., at levels of occupancy described), such that a partition of the set of partitions in a representative state contains a single nucleic acid molecule of the set of nucleic acid molecules; amplifying the set of nucleic acid molecules within individual partitions of the set of partitions for a set of amplification cycles, such that the partition in the representative state contains a set of amplicons generated from the single nucleic acid molecule; performing a nucleic acid purification operation upon amplicons of partitions of the set of partitions; and returning a prepared nucleic acid library upon performance of the nucleic acid purification operation. The library preparation operations can have amplification bias, artifact sequence generation, and low false positive rates achievable, as described.

The computer system 601 can additionally or alternatively perform other aspects of digital multiplexed assays for characterizations involving other loci of interest, with applications of use described above.

The computer system 601 can regulate various aspects of analysis, calculation, and generation of the present disclosure, such as, for example, generating a plurality of partitions (e.g., from an aqueous mixture including sample material and materials for an amplification reaction) within a collecting container at a desired rate, transmitting heat to and from the plurality of partitions within the collecting container, performing an optical interrogation operation with the plurality of partitions within the collecting container, and/or performing one or more digital multiplexed assay operations. The computer system 601 can be an electronic device of a user or a computer system that is remotely located with respect to the electronic device. The electronic device can be a mobile electronic device.

The computer system 601 includes a central processing unit (CPU, also “processor” and “computer processor” herein) 605, which can be a single core or multi core processor, or a plurality of processors for parallel processing. The computer system 901 also includes memory or memory location 610 (e.g., random-access memory, read-only memory, flash memory), electronic storage unit 615 (e.g., hard disk), communication interface 620 (e.g., network adapter) for communicating with one or more other systems, and peripheral devices 625, such as cache, other memory, data storage and/or electronic display adapters. The memory 610, storage unit 615, interface 620 and peripheral devices 625 are in communication with the CPU 605 through a communication bus (solid lines), such as a motherboard. The storage unit 615 can be a data storage unit (or data repository) for storing data. The computer system 601 can be operatively coupled to a computer network (“network”) 630 with the aid of the communication interface 620. The network 630 can be the Internet, an internet and/or extranet, or an intranet and/or extranet that is in communication with the Internet.

In some embodiments, the network 630 is a telecommunication and/or data network. The network 630 can include one or more computer servers, which can enable distributed computing, such as cloud computing. For example, one or more computer servers may enable cloud computing over the network 630 (“the cloud”) to perform various aspects of analysis, calculation, and generation of the present disclosure, such as, for example, generating a plurality of droplets within a collecting container at a predetermined rate or variation in polydispersity. Such cloud computing may be provided by cloud computing platforms such as, for example, Amazon Web Services (AWS), Microsoft Azure, Google Cloud Platform, and IBM cloud. In some embodiments, the network 630, with the aid of the computer system 601, can implement a peer-to-peer network, which may enable devices coupled to the computer system 601 to behave as a client or a server.

The CPU 605 may comprise one or more computer processors and/or one or more graphics processing units (GPUS). The CPU 605 can execute a sequence of machine-readable instructions, which can be embodied in a program or software. The instructions may be stored in a memory location, such as the memory 610. The instructions can be directed to the CPU 605, which can subsequently program or otherwise configure the CPU 605 to implement methods of the present disclosure. Examples of operations performed by the CPU 605 can include fetch, decode, execute, and writeback.

The CPU 605 can be part of a circuit, such as an integrated circuit. One or more other components of the system 601 can be included in the circuit. In some embodiments, the circuit is an application specific integrated circuit (ASIC).

The storage unit 615 can store files, such as drivers, libraries and saved programs. The storage unit 615 can store user data, e.g., user preferences and user programs. In some embodiments, the computer system 601 can include one or more additional data storage units that are external to the computer system 601, such as located on a remote server that is in communication with the computer system 601 through an intranet or the Internet.

The computer system 601 can communicate with one or more remote computer systems through the network 630. For instance, the computer system 601 can communicate with a remote computer system of a user. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 601 via the network 630.

Methods as described herein can be implemented by way of machine (e.g., computer processor) executable code stored on an electronic storage location of the computer system 601, such as, for example, on the memory 610 or electronic storage unit 615. The machine executable or machine-readable code can be provided in the form of software. During use, the code can be executed by the processor 605. In some embodiments, the code can be retrieved from the storage unit 615 and stored on the memory 610 for ready access by the processor 605. In some situations, the electronic storage unit 615 can be precluded, and machine-executable instructions are stored on memory 610.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Embodiments of the systems and methods provided herein, such as the computer system 601, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, or disk drives, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine-readable medium, such as computer-executable code, may take many forms, including a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 601 can include or be in communication with an electronic display 635 that comprises a user interface (UI) 640 for providing, for example, a visual display indicative of stages of or results from library preparation, with metrics associated with library characteristics. Generating libraries can include: generate nucleic acid libraries in a highly accurate manner, by enabling one or more of: distributing a sample comprising a set of nucleic acid molecules across a set of partitions at low occupancy (e.g., at levels of occupancy described), such that a partition of the set of partitions in a representative state contains a single nucleic acid molecule of the set of nucleic acid molecules; amplifying the set of nucleic acid molecules within individual partitions of the set of partitions for a set of amplification cycles, such that the partition in the representative state contains a set of amplicons generated from the single nucleic acid molecule; performing a nucleic acid purification operation upon amplicons of partitions of the set of partitions; and returning a prepared nucleic acid library upon performance of the nucleic acid purification operation, as described. Examples of UIs include, without limitation, a graphical user interface (GUI) and web-based user interface.

Methods and systems of the present disclosure can be implemented by way of one or more algorithms. An algorithm can be implemented by way of software upon execution by the central processing unit 605. The algorithm can, for example, generate a plurality of droplets within a collecting container with desired characteristics in relation to partitioned material, amplify the set of nucleic acid molecules within individual partitions of the set of partitions according to instructions provided; perform a nucleic acid purification operation upon amplicons of partitions of the set of partitions; and return a prepared nucleic acid library upon performance of the nucleic acid purification operation, as described.

### 5. Conclusions

The FIGURES illustrate the architecture, functionality and operation of possible implementations of systems, methods and computer program products according to preferred embodiments, example configurations, and variations thereof. In this regard, each block in the flowchart or block diagrams may represent a module, segment, or portion of code, which comprises one or more executable instructions for implementing the specified logical function(s). It should also be noted that, in some alternative implementations, the functions noted in the block can occur out of the order noted in the FIGURES. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts, or combinations of special purpose hardware and computer instructions.

It should be understood from the foregoing that, while particular implementations have been illustrated and described, various modifications may be made thereto and are contemplated herein. It is also not intended that the invention be limited by the specific examples provided within the specification. While the invention has been described with reference to the aforementioned specification, the descriptions and illustrations of the preferable embodiments herein are not meant to be construed in a limiting sense. Furthermore, it shall be understood that all aspects of the invention are not limited to the specific depictions, configurations or relative proportions set forth herein which depend upon a variety of conditions and variables. Various modifications in form and detail of the embodiments of the invention will be apparent to a person skilled in the art. It is therefore contemplated that the invention shall also cover any such modifications, variations and equivalents. It is intended that the following claims define the scope of the invention and that methods and structures within the scope of these claims and their equivalents be covered thereby.

As a person skilled in the art will recognize from the previous detailed description and from the figures and claims, modifications and changes can be made to the preferred embodiments of the invention without departing from the scope of this invention defined in the following claims.

What is claimed is:

1. A method comprising:
generating a prepared nucleic acid library, wherein the prepared nucleic acid library comprises a percentage of chimeric sequences below 10% upon performing a set of operations, and wherein the set of operations comprises:
distributing a sample comprising a set of nucleic acid molecules across a set of droplets of an emulsion, wherein greater than 80% of droplets of the set of droplets containing at least one nucleic acid molecule are in a representative state, and wherein a droplet of the set of droplets in the representative state contains a single nucleic acid molecule of the set of nucleic acid molecules;
amplifying the set of nucleic acid molecules within individual droplets of the set of droplets for a set of amplification cycles, such that the droplet in the representative state contains a set of amplicons generated from only the single nucleic acid molecule;
performing a nucleic acid purification operation upon amplicons of droplets of the set of droplets; and
returning the prepared nucleic acid library upon performance of the nucleic acid purification operation.

2. The method of claim 1, wherein at least 30% of the set of droplets contain no nucleic acid molecules.

3. The method of claim 1, wherein the set of droplets comprises at least 500,000 droplets, and wherein distributing the sample comprises generating the set of droplets at a rate of at least 200,000 droplets per minute.

4. The method of claim 1, wherein distributing the sample comprises driving the sample through a membrane comprising a distribution of holes, the membrane coupled to a reservoir outlet of a reservoir for the sample, and the reservoir aligned with the collecting container.

5. The method of claim 4, wherein driving the sample through the membrane comprises spinning the sample within the reservoir, the membrane, and the collecting container within a centrifuge.

6. The method of claim 1, wherein said amplifying comprises performing at least 20 amplification cycles of a polymerase chain reaction (PCR) protocol.

7. The method of claim 1, wherein said amplifying comprises performing an isothermal amplification protocol with amplification at 30° C. for at least 10 hours.

8. The method of claim 1, further comprising disrupting the emulsion with a butanol reagent, thereby releasing said amplicons from the set of droplets prior to performing the nucleic acid purification operation.

9. The method of claim 1, wherein the sample comprises T-cell receptor material, and wherein the set of nucleic acid molecules comprises variable, diversity, and joining (VDJ) sequences.

10. The method of claim 1, wherein the sample comprises ribosomal RNA (rRNA) of a microbial material sample, and wherein the set of nucleic acid molecules comprises at least one of 16S and ITS rRNA sequences.

11. The method of claim 1, wherein the sample comprises materials from single cells, and wherein the set of nucleic acid molecules comprises single cell whole genome sequences.

12. The method of claim 1, wherein the prepared nucleic acid library comprises an amplification bias level below a first threshold level, and wherein the set of operations further comprises:
determining first relative distribution values of a subset of input molecules of the set of nucleic acid molecules prior to said amplifying; and
determining second relative distribution values of amplicons of the subset of input molecules after said amplifying,
wherein the first threshold level represents a less than 5% difference between said first relative distribution values and said second relative distribution values.

13. A method comprising:
generating a prepared nucleic acid library, wherein the prepared nucleic acid library comprises an amplification bias level below a first threshold level upon performing a set of operations, and wherein the set of operations comprises:
distributing a sample comprising a set of nucleic acid molecules across a set of droplets of an emulsion, wherein greater than 80% of droplets of the set of droplets containing at least one nucleic acid molecule are in a representative state, and wherein a droplet of the set of droplets in the representative state contains a single nucleic acid molecule of the set of nucleic acid molecules;
amplifying the set of nucleic acid molecules within individual droplets of the set of droplets, such that the droplet in the representative state contains a set of amplicons generated only from the single nucleic acid molecule;
performing a nucleic acid purification operation upon amplicons of droplets of the set of droplets;
returning the prepared nucleic acid library upon performance of the nucleic acid purification operation;
determining first relative distribution values of a subset of input molecules of the set of nucleic acid molecules prior to said amplifying; and
determining second relative distribution values of amplicons of the subset of input molecules after said amplifying,
wherein the first threshold level represents a less than 5% difference between said first relative distribution values and said second relative distribution values.

14. The method of claim 13, wherein distributing the sample comprises driving the sample through a membrane comprising a distribution of holes, the membrane coupled to a reservoir outlet of a reservoir for the sample, and the reservoir aligned with the collecting container, and wherein driving the sample fluid through the membrane comprises spinning the reservoir with the sample, the membrane, and the collecting container within a centrifuge.

15. The method of claim 13, wherein said amplifying comprises one of: a) performing at least 20 amplification cycles of a polymerase chain reaction (PCR) protocol, and b) performing an isothermal amplification protocol with amplification at 30° C. for at least 10 hours.

16. The method of claim 13, further comprising disrupting the emulsion, thereby releasing said amplicons from the set of droplets prior to performing the nucleic acid purification operation.

17. The method of claim 16, wherein disrupting the emulsion comprises at least one of: mixing the emulsion with a butanol reagent and mechanically disrupting the emulsion.

18. The method of claim 13, wherein the sample comprises T-cell receptor material, and wherein the set of nucleic acid molecules comprises variable, diversity, and joining (VDJ) sequences, such that the prepared nucleic acid library is an immune repertoire sequence library.

19. The method of claim 13, wherein the sample comprises ribosomal RNA (rRNA) of a microbial material sample, and wherein the set of nucleic acid molecules comprises at least one of 16S and ITS rRNA sequences, such that the prepared nucleic acid library is an rRNA sequence library.

20. The method of claim 13, wherein the sample comprises materials from single cells, and wherein the set of nucleic acid molecules comprises single cell whole genome sequences, such that the prepared nucleic acid library is single cell whole genome sequence library.
